(19) United States
(12) Patent Application Publication
(10) Pub. No.: US 2011/0312893 A1
(43) Pub. Date: Dec. 22, 2011

(54) METHOD FOR ISOLATING NEURAL CELLS WITH TENASCIN-R COMPOUNDS

(75) Inventors: Penka Pesheva, Bonn (DE); Rainer Probstmeier, Bonn (DE)

(73) Assignee: RHEINISCHE -FRIEDRICH-WILHELMS-UNIVERSITÄT BONN, Bonn (DE)

(21) Appl. No.: 13/120,474

(22) PCT Filed: Sep. 16, 2009
(86) PCT No.: PCT/EP2009/062024

§ 371 Date: Aug. 24, 2011

(30) Foreign Application Priority Data
Sep. 23, 2008 (EP) 08164921.2

(51) Int. Cl. A61K 38/17; C07K 19/00; C07H 21/04; C12N 15/63; C12P 21/02; C07K 16/18; C12N 5/16; C12N 5/079; C12N 1/21; C12N 1/19; C12N 1/15; C12N 5/10; A61P 25/00; A61P 25/28; G01N 33/577; C07K 14/47

(52) U.S. Cl. 514/17.7; 530/350; 536/23.5; 435/320.1; 435/69.1; 435/69.7; 530/387.9; 435/331; 435/377; 435/252.33; 435/254.2; 435/254.11; 435/325; 435/348; 435/369; 435/378; 435/375; 435/7.1

(57) ABSTRACT

The invention relates to a method for isolating neural cells using tenascin-R compounds, tenascin-R fragments particularly suited for said method and tenascin-R fusion proteins, to recombinant preparation of said tenascin-R compounds, and to a kit for carrying out said method and to the use of said method for preparing high-purity neural cell populations. The invention further relates to antibodies suitable for detecting and isolating tenascin-R compounds.

The invention relates to a process for isolating neural cells using tenascin-R compounds, tenascin-R fragments and tenascin-R fusion proteins that are particularly suitable for such process, the recombinant preparation of such tenascin-R compounds, and a kit for performing this process, and the use of the process for preparing highly pure neural cell populations. The invention further relates to antibodies suitable for the detection and isolation of tenascin-R compounds.

### BACKGROUND OF THE INVENTION

When cells come into contact with the surrounding extracellular environment, then cell may show a wide variety of responses that may range from rejection and avoidance of the environment on the one hand to stable cell adhesion on the other. The interplay between components of the extracellular environment, the extracellular matrix, on the one hand and receptors for such components present on the cell surface on the other hand represents the basis of many processes of development biology, which are characterized for a cell, inter alia, by proliferation, migration or differentiation behavior. Such cellular behavior leads to pattern formation on the organism level, or to new formation as a result of injury on the tissue or organ level (Boudreau, N.J., Jones, P. L., Biochem. J. 339: 481-488 (1999); Sobeih, M. M., Corfas, G., Int. J. Dev. Neurosci. 148: 971-84 (2002); Schmid, R. S., Anton, E. S., Cereb. Cortex. 13: 219-24 (2003)). In the central nervous system (CNS), the regulated expression of extracellular matrix components, such as chondroitin sulfate proteoglycans or proteins of the tenascin family, correlates with biological processes which include both the adhesion and migration of neurons, the navigation of axons, synapse formation and plasticity, the action of growth factors and cytokines as well as the survival of neurons and the structural organization of the extracellular matrix (Dow, K. E., Wang, W., Cell Mol. Life Sci. 54: 567-81 (1998); Wright, J. W. et al., Peptides 23: 221-46 (2002); Grimpe, B., Silver, J., Prog. Brain Res. 137: 333-49 (2002); Bosman, F. T., Stamenkovic, I., J. Pathol. 200: 423-8 (2003)).

Tenascin-R (TN-R) (formerly referred to as J1-160/180, janusin or restrictin) is a member of the tenascin family of extracellular matrix proteins that occurs exclusively in the CNS of vertebrates, where it is expressed by oligodendrocytes and some groups of neurons, i.e., motoneurons and interneurons, during later stages of the development and in the adult state (Pesheva, P., Probstmeier, R., Prog. Neurobiol. 61: 465-93 (2000); Scherberich, A. et al., J. Cell Sci. 117: 571-81 (2004)). The protein occurs in two molecular forms having molecular weights of 160 kD (TN-R 160) or 180 kD (TN-R 180). TN-R is found in the tissue primarily in association with oligodendrocytes, myelinized axons, perineuronal networks of motoneurons and interneurons, and in regions rich in dendrites and synapses.

TN-R is composed of four different domain structures. The N terminus, whose sequence occurs only in tenascin proteins, contains a cysteine-rich segment (Cys-rich) and is followed by four and a half EGF-like segments (EGF-like) as well as 9 fibronectin type III (FN III) like domains (of which the 6th domain may be alternatively spliced). The C terminus of the TN-R protein is formed by a globular fibrinogen-like domain (FNG). Single TN-R polypeptide chains are connected through disulfide bridges at their N termini and thus form homo-trimers (TN-R 180) or dimers (TN-R 160), the latter being formed by the proteolytic cleavage of TN-R 180 near the N terminus (Woodworth, A. et al., J. Biol. Chem. 279: 10413-21 (2004)).

The functional range of TN-R comprises the molecular control of neural cell adhesion, migration and differentiation (from the axon navigation of neurons forming processes to the maturation of myelin-forming oligodendrocytes) during normal development-biological processes as well as regenerative processes upon injury in the adult brain (Pesheva, P., Probstmeier, R., Prog. Neurobiol. 61: 465-93 (2000); Chiquet-Ehrismann, R., Int. J. Biochem. Cell. Biol. 36: 986-90 (2004)). Inter alia, TN-R acts as an adhesive or anti-adhesive molecule, as a differentiation factor for oligodendrocytes, or as a “stop molecule” for growing axons. These properties depend on the corresponding general conditions: the respective cell type, the presence of cellular receptors and signal cascades, the distribution in space and the posttranslational modification of the TN-R glycoprotein. Some of the cellular receptors of TN-R that have been identified (F3/F11, disialogangliosides, sulfatides) induce different cellular mechanisms of action which are important on the one hand in the pattern formation during ontogenesis and on the other hand in regenerative processes (Angelov, D. et al., J. Neurosci. 18: 6218-29 (1998); Probstmeier, R. et al., J. Neurosci. Res. 60: 21-36 (2000); Montag-Sallaz, M., Montag, D., Genes Brain Behav. 2: 20-31 (2003); Saghatelyan, A. et al., Nat. Neurosci. 7: 347-56 (2004); Brenneke, F. et al., Epilepsy Res. 58: 133-43 (2004)). The two essential effects of TN-R on the behavior of neural cells are the inhibition of cell adhesion and neurite growth on the one hand, and the promotion of oligodendrocyte adhesion and differentiation on the other. The latter is mediated by sulfatides and ultimately causes oligodendrocyte migration and myelinization/remyelinization. The former can occur either substrate-independently (mediated by F3/F11 and other, as yet unknown factors) or substrate/integrin-dependently (mediated by fibronectin and GD2/GD3). The inhibitory effect of TN-R ultimately has an influence on the neural cell migration, the space-coordinated axon growth and synaptogenesis, or it contributes to the prevention of axon regeneration and the adhesion of activated microglia in a TN-R-rich environment under injury conditions.

Some neural receptors and intracellular signal pathways that mediate the effect of TN-R in neuronal and glial cells are known, and molecular components involved in the expression of TN-R by oligodendrocytes and motoneurons could also be identified (Table 1; Pesheva, P. et al., Prog. Brain Res. 132: 103-14 (2001)).

TABLE 1
Cellular receptors and ligands of the extracellular
matrix identified for TN-R.

| | Tenascin-R | Ligands of the | Tenascin-R |
|---|---|---|---|
| Cellular | binding | extracellular | binding |
| receptors | domain | matrix | domain |
| F3/F11 | EGF-L, FN2-3 | Fibronectin | CS GAG, unknown |
| β2 subunit of | FN1-2, FN6-8 | Collagens | unknown |
| Na channels | | | |
| Neurofascin | FN2-5 | Tenascin-C | CS GAG, |
| | | | Ca²⁺-dependent |
| CALEB | FNG | Tenascin-R | unknown, |
| | | | cation²⁺-dependent |
| MAG | EGF-L, FNG | Lecticane | FN3-5, Ca²⁺-dependent |
| Sulfatides | unknown | Phosphacane | EGF-L, Ca²⁺-dependent |
| Gangliosides | unknown | | |

In the right-hand half of the Table, the domains of TN-R relevant for the respective interaction are stated; CS GAG: chondroitin sulfate glycosaminoglycans; EGF-L: EGF-like segments and cystein-rich segment.

As TN-R is an extracellular matrix protein constituted of several domains, an attribution of the distinct biological functions to distinct domain regions and/or distinct glycostructures of TN-R suggests itself. Similar to the other known TN-R proteins, the human TN-R protein is composed of different distinct domains (Carnemolla et al., 1996). Starting with a cysteine-rich region at the N terminus, followed by 4.5 EGF-like domains and 9 FNIII-like domains (of which the 6th domain can be alternatively spliced), the molecule ends at the C terminus with a fibrinogen-like domain. The published human TN-R mRNA sequence comprises 4716 bases with 9 FNIII-like domains. Of these, the coding region comprises the segment between bases 82 to 4158, the signal peptide (for the secretion of the TN-R protein) comprises the region of bases 82 to 150 (SEQ ID No. 2).

It is known that TN-R proteins purified from adult rodent brain promote the adhesion and process formation of O4/sulfatide-positive oligodendrocytes isolated from early postnatal brain (Pesheva, P. et al., J. Neurosci. 17: 4642-51 (1997)).

These processes are mediated by sulfatides, a group of glycolipids occurring in the cell membrane of oligodendrocytes. An important consequence of the interaction of TN-R with sulfatide-expressing oligodendrocytes is a stimulation of the maturation of these cells; i.e., the increased expression of myelin-specific proteins and glycolipids, which suggests a sulfatide-mediated mode of action of the differentiation potential of TN-R on oligodendrocytes (Pesheva, P. et al., J. Neurosci. 17: 4642-51 (1997)).

EP 0 759 987, U.S. Pat. No. 5,635,360, U.S. Pat. No. 5,681,931 and U.S. Pat. No. 5,591,583 describe human TN-R and its immunological detection using antibodies directed against a protein fragment that corresponds to the nucleotides 2686-3165 of the cDNA sequence and thus to the FN III domains 6 and 7 of human TN-R.

The earliest stages of developing glia cells in mammals are found in ventral regions of the neural tube in the spinal cord, and in ventricular zones of the fore-brain in the brain. In these regions, first precursor cells of oligodendrocytes characterized by a simple morphology and the expression of the disialogan-glioside GD3 and/or of O4 antigens proliferate and migrate in the following time into regions of the later formed white substance (Miller, R. H. Prog. Neurobiol. 67: 451-67 (2002); Noble, M. et al., Dev. Biol. 265: 33-52 (2004); Liu, Y. Rao, M. S., Biol. Cell. 96: 279-90 (2004)). There, the cells become postmitotic and differentiate into mature oligodendrocytes with a complex morphology. This maturation process correlates with the expression of myelin-specific lipids (sulfatides and galactocerebrosides) and proteins (MBP, MAG and PLP). The formation, the survival and the differentiation of oligodendrocytes in myelin-forming cells are regulated through various growth factors (bFGF, PDGF, CNTF, IGF-1, NT-3), hormones and extracellular matrix molecules (thyroid hormones, retinolic acid, TN-R) (Dubois-Dalcq, M., Murray, K., Pathol. Biol. (Paris) 48: 80-6 (2000); Kagawa, T. et al., Microsc. Res. Tech. 52: 740-5 (2001); Noble, M. et al., Dev. Neurosci. 25: 217-33 (2003)).

Since especially for glial cells (such as oligodendrocytes and neural stem cells) no adequate model systems in the form of cell lines exist, relatively time-consuming and low-efficient enrichment processes of primary cells have had to be recurred to to date in their recovery both in basic research and within the scope of potential diagnostic/therapeutic fields of application. In addition, these processes often enable only the preparation of enriched mixed cell populations. The previous processes for isolating defined cell populations utilize different techniques, such as density gradient centrifugations or immunological processes (Fluorescence-activated cell sorting, biomagnetic cell sorting, antibody- and complement- mediated cell killing, antibody panning) (Luxembourg, A. T. et al., Nat. Biotechnol. 16: 281-5 (1998); Uchida, N. et al., Proc. Natl. Acad. Sci. 97: 14720-5 (2000); Nistri, S. et al., Biol. Proced. Online 4: 32-37 (2002); Nunes, M. C. et al., Nat. Med. 9: 439-47 (2003); Vroemen, M., Weidner, N., J. Neurosci. Methods 124: 135-43 (2003)). Such methods are not very efficient and result in an incomplete enrichment (by density gradient centrifugations, for example, enrichments of distinct cell populations are only possible), or they are time-intensive and/or accompanied by high cell losses (as in immunological processes). This applies, in particular, to oligodendrocytes which to date have been obtained by several weeks of cultivation of mixed glial cultures (McCarthy, K. D., DeVellis, J., J. Cell Biol. 85: 890-902 (1980); Kramer, E. M. et al., J. Biol. Chem. 274: 29042-9 (1999); Testai, F. D. et al., J. Neurosci. Res. 75: 66-74 (2004)) or by several selection steps by means of fluorescence-activated/biomagnetic cell sorting or antibody panning (Scarlato, M. et al., J. Neurosci. Res. 59: 430-5 (2000); Tang, D. G. et al., J. Cell Biol. 148: 971-84 (2000); Diers-Fenger, M. et al., Glia 34: 213-28 (2001); Crang, A. J. et al., Eur. J. Neurosci. 20: 1445-60 (2004)).

WO2006/067094 describes purified TN-R proteins that support the stable adhesion of oligodendrocytes in different stages of maturity and have an anti-adhesive effect on neuronal and microglial cells. This enables the isolation and purification of defined cell populations from neural primary tissue, especially for the direct selective purification of oligodendrocytes from mixed neural cell populations. A process is herein provided that enables the isolation of a highly pure defined cell population, especially an oligodendrocyte population, from primary tissue of neural origin in a single purification step. The purification of the cells is effected by a selection step from a single cell suspension by means of a probe containing tenascin-R comprising tenascin-R compounds selected from native tenascin-R (briefly “TN-R” hereinafter) as well as homologues and fragments thereof and fusion proteins of such compounds. A preferred tenascin-R fragment contains the C terminus of native tenascin-R or a substitution, deletion and/or addition mutant thereof, especially the region encoded by the nucleotides 3439-4155 of SEQ ID No. 1.

It has now been found that a fragment comprising amino acid residues 24 to 189 of SEQ ID No. 2 of human TN-R or a homologue or fragment thereof, such as the fragment H-TN-R-Cys, which includes amino acid residues 24 to 189 of SEQ ID No. 2, can be applied in a one-step process for the isolation of highly pure glial, especially oligodendroglial, cell populations with similar suitability as the H-TN-R-S3 fragment with amino acid residues 1120 to 1358 of SEQ ID No. 2 as described in WO 2006/067094. In contrast to H-TN-R-S3, the H-TN-R-Cys fragment additionally binds sphingomyelin (a phospholipid occurring in the cell membrane of different cell types and involved in cellular signal mechanisms) and supports the survival and the transdifferentiation of adult human mesenchymal stem cells into neuronal or oligodendroglial cells.

Thus, the invention relates to:

(1) a process for the isolation and purification of neural cells from neural primary tissue of vertebrates, comprising the selection of the cells from a single cell suspension by means of a probe containing tenascin-R (“tenascin-R probe”), which includes an N-terminal fragment of native tenascin-R (TN-R) as well as homologues and fragments thereof and fusion proteins of such compounds;

(2) a preferred embodiment of (1) wherein said tenascin-R fragment is a peptide with the sequence of amino acid residues 24-189 of SEQ ID No. 2 that enables the survival and/or transdifferentiation of adult human mesenchymal stem cells into neuronal or oligodendroglial cells, especially under defined serum-free culturing conditions;

(3) a tenascin-R fragment or tenascin-R fusion protein as defined above under (1) or (2);

(4) a DNA which codes for a tenascin-R fragment or TN-R fusion protein according to (3);

(5) a vector which comprises a DNA according to (4);

(6) a host organism transformed/transfected with a vector according to (5) and/or having a DNA according to (4);

(7) a process for preparing a tenascin-R fragment or TN-R fusion protein according to (3), comprising the step of culturing said host organism according to (6);

(8) an antibody, especially a monoclonal antibody, obtainable by the immunization of a suitable host organism with a tenascin-R fragment according to (3);

(9) a cell line or hybridoma cell line which produces a monoclonal antibody according to (8);

(10) a kit for the isolation and purification of neural cells, especially of oligodendrocytes, according to the process of (1) or (2), especially containing

(i) a tenascin-R probe as defined in (1) or (2); and/or

(ii) a vector which codes for the tenascin-R probe defined in (i); and/or

(iii) a stock culture of a cell line which is suitable for expressing said tenascin-R probe as defined in (1) or (2);

(11) the use of a tenascin-R fragment or tenascin-R fusion protein as defined in (3) for obtaining neural cells, especially oligodendrocytes, for growing differentiated cells, especially neural cells, in neurobiological and cell-physiological examinations, in biological and clinical research and for diagnostic and therapeutic processes in vitro and in vivo, especially for the preparation of a medicament for cell therapy and for the therapy of neurodegenerative diseases accompanied by a loss of oligodendrocytes or myelin, especially multiple sclerosis and periventricular leukomalacia (PVL);

(12) a process for preparing oligodendrocytes from isolated stem cells in vitro by incubating the stem cells in the presence of a tenascin-R fragment or tenascin-R fusion protein as defined in (3);

(13) a process for cell therapy or for the therapy of neurodegenerative diseases accompanied by a loss of oligodendrocytes or myelin, especially multiple sclerosis and periventricular leukomalacia (PVL), comprising the step of administering a tenascin-R fragment or tenascin-R fusion protein as defined in (3) to a human or animal patient;

(14) a process for the therapy and prophylaxis of traumatic nerve lesions or for selectively influencing the neural development, comprising the step of administering a pharmacologically sufficient amount of the antibody according to (8) to a human or animal patient in need of such treatment.

### BRIEF DESCRIPTION OF THE FIGURES

FIG. 1: Characterization of the specificity and functional activity of the mono-clonal antibody R4. A) Western blot analysis of affinity-purified H-TN-R-S3 and H-TN-R-Cys fragments with Tag-specific (V5) and TN-R-specific (R4) monoclonal antibodies. The recombinant fragments H-TN-R-S3 (S3) and H-TN-R-Cys (Cys) purified by nickel-agarose (20 ng/slot) were separated by gel electrophoresis, blotted onto nitrocellulose filters and incubated with V5 or R4 antibodies. Immuno-reactive protein bands were detected by incubation with peroxidase-coupled anti- mouse IgG antibodies. Arrows indicate the molecular weight of H-TN-R-S3 (40 kD) and H-TN-R-Cys (34 kD). B) Influence of polar glycolipids, lysenin (sphingomyelin-binding protein), O4- and TN-R-specific antibodies on TN-R-mediated cell adhesion. TN-R substrates (TN-R, H-TN-R-Cys, H-TN-R-S3) were preincubated in the absence (−GL/Ab) or presence of monosialogangliosides (+GM1), sulfatides (+Sulf) and TN-R-specific antibodies (R4, R6). Erythrocytes were sown in the presence or absence of lysenin (100 μg/ml) and O4 antibodies (+O4 Ab) onto the corresponding substrates. The number of adherent cells after one hour of incubation on untreated TN-R substrates (−GL/Ab) was set at 100% C) Interaction of R4 epitope bearing TN-R/TN-R fragments with polar glycolipids in solid-phase binding assay. Microtitration plates were coated with sulfatides (Sulf), galactocerebrosides (GalC), sphingomyelin (SM) or sialogangliosides (GM1, GD1a; all at 100 pmol/well), and incubated with TN-R or recombinant TN-R fragments (H-TN-R-Cys, H-TN-R-S3) at room temperature for 2 h. The binding of TN-R/TN-R fragments was detected with R6 (for TN-R) or V5 antibody (after 1.5 hours of incubation at RT) with peroxidase-coupled anti-mouse IgG antibodies. The maximum binding to sulfatides was set at 100% for the respective protein.

FIG. 2: Selection of oligodendrocytes from single cell suspensions (from brain tissue of 2 days old mice) for substrate-bound TN-R fragments (H-TN-R-Cys, H-TN-R-S3). Cells growing on these substrates were cultured for 3 days in serum-free growth medium (3 div, upper pictures) and for another 3 days (6 div, lower pictures) in serum-free differentiation medium. Adherent cells could be identified as oligodendrocytes by means of indirect immunofluorescence staining with sulfatide-specific antibody (O4). 99±1% of the isolated cells were O4-positive. Survey (10×) and detail pictures (20× and 40×) show O4-positive cells on the different substrates after different culturing times.

FIG. 3: H-TN-R-Cys-mediated neural differentiation of human mesenchymal stem cells (hMSC) from adult bone marrow. Cells growing on the different substrates were stained with antibodies against marker molecules for neurons (βIII tubulin), neural/glial progenitor cells (nestin), oligodendrocytes (sulfatide, GalC) or astrocytes (GFAP) by means of indirect immunofluorescence after the stated culturing times. To determine the cell survival, adherent cells on the different substrates were stained with DAPI at the beginning and end of the culturing period, and the ratio of the corresponding total cell numbers was calculated. A) hMSC (left picture) were cultured on substrate-bound laminin and TN-R fragments (H-TN-R-Cys, H-TN-R-S3) for 5 days in serum-free growth medium. B) Glial differentiation of hMSC on substrate-bound TN-R fragments (H-TN-R-Cys, H-TN-R-S3). Cells plated onto TN-R fragments or laminin were cultured for 2 days in serum-free growth medium and for another 3 days in serum-free glial differentiation medium. The ratio of the number of antibody-marked cells on the respective substrate to the total cell number (100%) was calculated. More than 80% of the cells on H-TN-R-Cys substrates were sulfatide/GalC-positive and could be identified as oligodendrocytes. C) Neuronal differentiation of hMSC on substrate-bound TN-R fragments (H-TN-R-Cys, H-TN-R-S3). Neurosphere-derived cells plated onto TN-R fragments or poly-D-lysine (PDL) were cultured for 10 days in serum-free neuronal differentiation medium. The ratio of the number of antibody-marked cells on the respective substrate to the total cell number (100%) was calculated. 80% of the cells on H-TN-R-Cys substrates were βIII-tubulin-positive and could be identified as neurons. D) Conclusion relating to the influence of H-TN-R-Cys on the neural transdifferentiation of hMSC under defined serum-free culturing conditions. Survival rate and expression of neuron-(βIII-tubulin) and oligodendrocyte-specific (sulfatide/GalC) molecules by cells grown on H-TN-R-Cys substrates and cultured in glial or neuronal differentiation medium.

FIG. 4: CLUSTAL W (1.82) alignment of the known amino acid sequences of tenascin-R from different vertebrates, namely house mouse (SEQ ID No. 6), rat (SEQ ID No. 7), human (SEQ ID No. 8), chicken (SEQ ID No. 9) and zebra fish (SEQ ID No. 10), “*” designates identical amino acids, “:” designates a conservative amino acid exchange, “.” designates a semi-conservative amino acid exchange.

Sequence Listing—Free Text

| SEQ ID No. | Description |
|---|---|
| 1 and 2 | TN-R nucleic acid sequence and protein |
| 3-4 | primer |
| 5 | vector pIN-II-omp A2 |
| 6-10 | TN-R protein from house mouse, rat, human, |
| | chicken and zebra fish |

### DETAILED DESCRIPTION OF INVENTION

The invention relates to a process for isolating highly pure cell populations from neural primary tissue in a one-step process by means of native tenascin-R proteins or TN-R fragments of vertebrates, preferably fish, amphibians, reptiles, birds and mammals, more preferably shark, carp, chicken, rodents including mouse and rat, cattle, pig and human, even more preferably rodents and humans, by selective substrate adhesion.

Phylogenetic studies relating to the function of tenascin-R in the nerve system of vertebrates show the support of adhesion and process formation in oligodendrocytes as a function of tenascin-R proteins that is highly conserved in evolution. The present invention shows that this property of the total tenascin-R protein can also be localized on distinct recombinant fragments of human tenascin-R protein.

In the context of the present invention, “tenascin-R probe” means an N-terminal fragment of native tenascin-R recognized and bound by oligodendrocytes in vivo and in vitro, and/or further tenascin-R compounds including tenascin-R homologues and tenascin-R fragments that are also bound with high sensitivity and specificity in vivo and in vitro by oligodendrocytes and/or other neural cells from primary tissue, wherein “tenascin-R compounds” has the meaning as defined elsewhere. This also includes fusion proteins of several tenascin-R compounds, preferably from two or three tenascin-R compounds. The components of these fusion proteins can be interconnected directly or through a (flexible) linker peptide. More preferably, the tenascin-R probe contains human tenascin-R fragments, even more preferably the partial sequence of human TN-R comprising the amino acid residues 24 to 189 of SEQ ID No. 2. In addition to its TN-R portions, the tenascin-R probe may also have non-TN-R portions which ensure the stability of the TN-R portion and/or maintain or increase the immobilizability and coupling ability to other molecules. In particular, a tenascin-R probe according to the invention is prepared recombinantly.

According to the invention, “tenascin-R compounds” are proteins or peptides which are either N-terminal fragments of native TN-R or their substantially identical homologues.

“Primary tissue” is a biological tissue which is taken directly from an organism and used without further modifying its genetic material. “Primary cells” are cells originating from primary tissue. Primary cells are advantageous over cell lines because they are closest to the cells in the intact organism structurally and functionally due to their origin.

In the case of a protein, “isolated” means that it has been separated or purified from other proteins with which it is normally associated in the organism in which it naturally occurs. This includes biochemically purified proteins, recombinantly prepared proteins and proteins synthesized on a chemical route. The definition also applies, mutatis mutandis, to nucleic acids, especially DNA, and peptides.

“Native” is used interchangeably with “natural” or “naturally occurring”.

For the “recombinant preparation” according to the invention, methods usual in the art for the recombinant preparation of eukaryotic proteins are used, especially the expression as a fusion protein in pro- and eukaryotic cells, more preferably the expression as a fusion protein having a polyhistidine tail. Further preferred is the use of DNA coding for the corresponding protein.

Within the scope of the present invention, nucleic acid sequences, especially DNA sequences coding for proteins or peptides according to the invention, are either identical or substantially identical with the native sequence or its underlying artificial sequence according to the invention. If a specific nucleic acid sequence is mentioned within the scope of the present invention, it includes this sequence itself and the sequences substantially identical therewith. “Substantially identical” means that only an exchange of bases in the sequence within the scope of the degenerate nucleic acid code has been effected, i.e., that the codons within coding sequences of the substantially identical nucleic acid are changed thereby as compared to the original molecule merely in a way that does not lead to a change of the amino acid sequence of the translational product (usually exchange of the codon by another codon of its codon family). Within the scope of the present invention, sequences which are mentioned in the Sequence Listing and their fragments according to the invention are preferred.

Protein sequences and peptide sequences can be modified within the scope of the present invention by the substitution of amino acids. Preferred are those substitutions in which the function and/or conformation of the protein or peptide is retained, more preferably those substitution in which one or more amino acids are replaced by amino acids which have similar chemical properties, e.g., alanine for valine (“conservative amino acid exchange”). The proportion of substituted amino acids as compared to the native protein or, if it is not a native protein, to the starting sequence is preferably 0-30% (based on the number of amino acids in the sequence), more preferably 0-15%, even more preferably 0-5%.

Nucleic acid sequences and amino acid sequences can be employed as full length sequences or as addition or deletion products of such full length sequences for performing the invention. In the amino acid sequences, the addition products also include fusion proteins and additionally amino acid sequences formed by the addition of 1-100, preferably 1-30, more preferably 1-10 amino acids. The added amino acids may be inserted or added singly or in contiguous segments of 2 or more interconnected amino acids. The addition may be effected at the N or C terminus or within the original sequence. Several additions in one sequence are allowed, wherein a single addition is preferred, more preferably an addition at the C or N terminus.

The deletion products of the full length amino acid sequences are formed, unless stated otherwise for specific sequences, by the deletion of 1-100, preferably 1-20, more preferably 1-10 amino acids. The deleted amino acids may be removed singly or in contiguous segments of 2 or more interconnected amino acids. The deletion may occur at the N or C terminus or within the original sequence. Several deletions in one sequence are allowed, wherein a single deletion is preferred, more preferably a deletion at the C or N terminus.

The allowable deletions and additions in the nucleic acid sequences according to the invention have the extent and nature that correspond to the allowable amino acid deletions or additions. Apart from the deletion and addition of entire codons, the addition or deletion of single bases or pairs of bases is also possible.

A fragment of a nucleic acid or protein is a part of its sequence that is shorter than the full length, but still contains a minimum sequence segment required for hybridization or specific binding. In the case of a nucleic acid, this sequence segment is still capable of hybridizing with the native nucleic acid under stringent conditions and preferably comprises at least 15 nucleotides, more preferably at least 25 nucleotides. In the case of a peptide, this sequence segment is sufficient to enable the binding of an antibody specific for a segment of the native protein or of a cell that binds to TN-R or a TN-R fragment. The peptide length is preferably at least 5 amino acids, more preferably at least 10 amino acids, even more preferably at least 20 amino acids.

A “fusion protein” in the context of the present invention comprises at least one tenascin-R compound according to the invention that is linked to at least one second protein or peptide. Such second protein or peptide is preferably a selection or marker protein, a protein that serves for the binding of the fusion protein to a surface, or a tenascin-R compound. Preferred are fusion proteins of native tenascin-R and further functional proteins and peptides, and fusion proteins of two or more, more preferably two or three, tenascin-R fragments. The nucleic acid sequences coding for the individual parts of the fusion protein in a vector or transformed host organism are connected with one another in a way that allows the expression under the control of a single promoter. The amino acid sequences of the individual functional parts of the fusion protein are linked to one another either directly or through a linker. The linker has a length of 1-30 amino acids, preferably 10-20 amino acids.

“Neurodegenerative diseases” are diseases of the nerve system associated with the dying of neuronal and/or macroglial cells due to impairment of their functional integrity. Such cell damages and losses lead to failure or impairment of the functions of the affected regions of the nerve system and/or the body parts controlled by these regions.

The cells isolated by the process according to embodiment (1) and (2) are preferably glial cells, more preferably oligodendrocytes.

The tenascin-R probe for use in embodiment (1) preferably originates from brain tissue of fish (shark, carp, goldfish, trout etc.), amphibians (salamander, frog etc.), reptiles (Greek tortoise, grass snake etc.), birds (chicken, pigeon etc.) and mammals (hedgehog, rabbit, rodent, pig, cattle, human), especially mammals, more especially rodent, pig, cattle or human, wherein the fragments correspond to the above defined human fragment. The TN-R protein fragment according to the invention is preferably prepared recombinantly, and/or is human TN-R fragments. In the latter case, a preferred source of the corresponding DNA sequence is the human neuroblastoma cell line SH-SY5Y. The expression of the fragments is preferably effected after transformation of human Flp-In 293 cells (Invitrogen) or in selected E. coli strains with the corresponding DNA sequences.

In embodiment (1), the TN-R probe may additionally contain further functional protein or peptide sequences and/or be coupled to a support.

In a preferred aspect of embodiments (1) and (2), the tenascin-R probe is a tenascin-R fragment (a) corresponding to the human tenascin-R fragment encoded by nucleotides 151-648 of SEQ ID No. 1, and preferably is a fragment with amino acid residues 24 to 189 of SEQ ID No. 2.

In another preferred aspect of embodiments (1) and (2), the tenascin-R probe (b) is a fragment of (a) in which up to 10, preferably up to 5, amino acid residues are cleaved off from the N and/or C terminus, and/or which has at least 150 amino acid residues.

In another preferred aspect of embodiments (1) and (2), the tenascin-R probe (c) is a substitution, deletion and/or addition mutant of (a) or (b).

In another preferred aspect of embodiments (1) and (2), the tenascin-R probe is a tenascin-R fusion protein (d) comprising a tenascin-R fragment as defined above under (a) to (c) and a functional component including one or more further functional peptides or proteins, or a tenascin-R fusion protein composed of two or more, preferably two or three, of the functional tenascin-R fragments as defined above under (a) to (c).

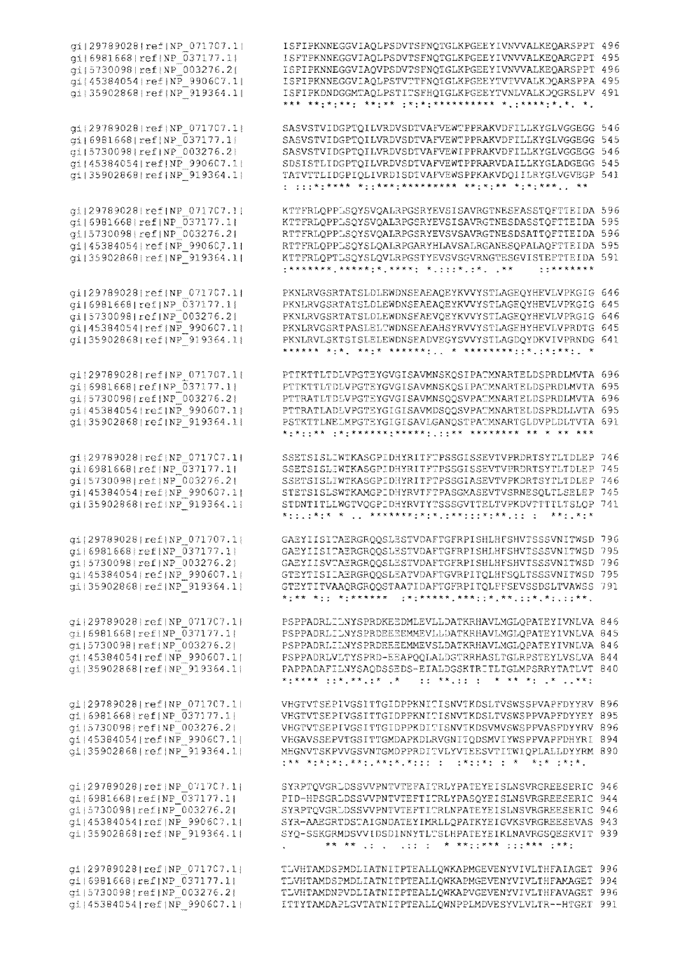

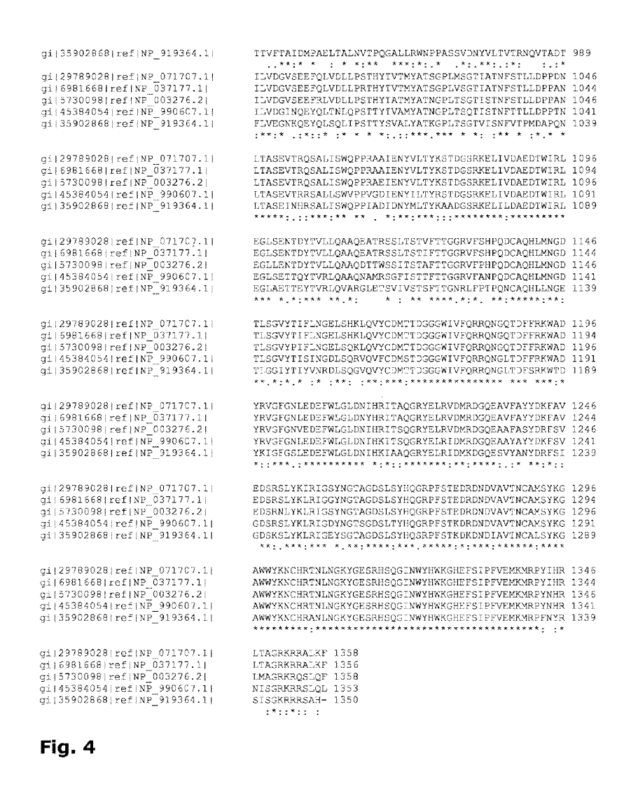

The phylogenetically conserved property of TN-R proteins, i.e., being able to act as an adhesive substrate for oligodendrocytes, is reflected by a high conservation of the amino acid sequence between different vertebrate species on the molecular level: the human TN-R sequence shows homologies of 93% (with rat), 75% (with chicken) and 60% (with zebra fish) (FIG. 4). This property is utilized by the present application.

The isolation of TN-R from natural sources is preferably effected by known chromatographic and/or immunological methods for protein purification, especially affinity chromatography on TN-R antibodies (see Example 1 of WO2006/067094). As a source of TN-R, tissues and single cell suspensions of higher and lower vertebrates may be used.

Recombinant methods for the preparation of the TN-R probe include the usual known methods for the transformation of prokaryotes and eukaryotes (as described, e.g., in G. Schrimpf (Ed.), Gentechnische Methoden, 3rd edition, Spektrum Akademischer Verlag (2002); Smith, C., The Scientist 12(3): 18 (1998); Unger, T., The Scientist 11(17): 20 (1997)). Suitable methods for the preparation of recombinant proteins or protein fragments comprise transfection or transformation methods based on different expression systems/vectors for prokaryotic (especially E. coli) and eukaryotic cells (yeast, fungi, insect and mammal cells). In order to produce functionally active recombinant proteins (i.e., those which are the most similar to the native protein after their folding/conformation and glycosylation), mammal cells are preferred as producers. The different expression vectors for mammal cells are mainly distinguished in the type of promoter (SV40, CMV, human EF1alpha, MMTV-LTR, MSV-LTR, RSV-LTR, etc.), kind of expression (transient, constitutive, inducible), induction mechanism, selective marker (antibiotic or drug resistance and/or co-expression of easily detected proteins) and elements for the subcellular targeting of the gene product (mitochondria, nucleus, secretion). Pro- and eukaryotic expression systems that express the foreign gene constitutively are preferred as producers of recombinant proteins/protein fragments.

In a preferred embodiment of (2) for the expression of human recombinant protein fragments according to the invention, defined PCR fragments of human TN-R are cloned by means of the TOPO TA cloning system (Invitrogen) into the pcsecTag/FRT/V5-His-TOPO vector (Invitrogen), which allows the constitutive expression and secretion of the desired protein fragment provided with a 6×His peptide at the C terminus in human Flp-In 293 cells (Invitrogen). In Flp-In cells, the plasmid pFRT/lacZeo (Invitrogen) is stably integrated, and the FRT region is specifically recognized by Flp recombinase. When the Flp 293 cells are simultaneously transfected with the pOG44 plasmid, which enables the expression of Flp recombinase, and the pcsecTag/FRT/V5-His-TOPO vector, which bears the base sequence of the desired protein fragment, an incorporation of the portions of the pcsecTag/FRT/V5-His-TOPO vector necessary for the preparation of a secreted protein fragment occurs at the FRT region. This enables the secretion of polyHis-bearing protein fragments into the cell culture supernatant and their purification by nickel chelate chromatography from collected cell culture supernatants.

Another aspect of embodiment (1) is the preparation of the TN-R fragments by chemical synthesis by the fragmentation of isolated TN-R or recombinantly, preferably recombinantly, according to embodiment (7). Suitable recombinant methods include the usual known methods for the transformation of prokaryotes and eukaryotes (as described, e.g., in G. Schrimpf (Ed.), Gentechnische Methoden, 3rd edition, Spektrum Akademischer Verlag (2002); Smith, C., The Scientist 12(3): 18 (1998); Unger, T., The Scientist 11(17): 20 (1997)). For this purpose, a host organism according to embodiment (6) can be used that is transformed or transfected with a vector which comprises the above defined DNA sequences coding for TN-R or TN-R fusion protein. In addition to the mentioned DNA sequences, such a vector may also contain functional sequences adapted to the host organism, such as promoters, leader sequences etc. For the chemical preparation of TN-R fragments and for the fragmentation of isolated TN-R, methods known in the art can be used, such as solid-phase peptide synthesis and enzymatic or mechanic fragmentation methods.

Another preferred aspect of embodiment (1) relates to a fusion protein comprising a tenascin-R component selected from native tenascin-R or tenascin-R fragments and fusion proteins of two or more tenascin-R fragments, and a functional component which comprises functional peptide or protein sequences. The components can be connected with one another directly or by means of a (flexible) linker peptide. Another aspect relates to the combination of two or more of the above defined tenascin-R components, especially tenascin-R fragments, in a way that does not correspond to the native amino acid sequence to form a tenascin-R probe according to the invention. The linking may also be direct or by means of a linker peptide. For the synthesis, the above mentioned vector systems can be used, wherein those cDNA sequences of parts of the TN-R sequence that are not adjacent in space are linked to one another through terminal restriction enzyme cleavage sites according to usual technical methods.

The tenascin-R probe according to embodiments (1) and (2) preferably comprises the partial sequence of human TN-R that corresponds to amino acid residues 24 to 189 in SEQ ID No. 2. In a preferred aspect of (1) to (4), the probe has one of the amino acid sequences of this group or is composed of 2 or more of the amino acid sequences of this group in one fusion protein, wherein the repetition of one or more of the sequences within the fusion protein is also possible. The invention also relates to the nucleic acids which comprise nucleic acid fragments coding for such proteins, preferably DNA sequences and cDNA.

In a preferred aspect of embodiment (2), the tenascin-R probe is a fragment of human tenascin-R and can be obtained either by preparation from cells of humans or by recombinant production. In particular, as a native TN-R, it is obtainable from cells of neural origin, more preferably from SH-SY5Y neuroblastoma cells. The recombinant preparation of the TN-R fragments, in particular, is preferably effected in correspondingly transformed human Flp-In 293 cells or selected E. coli strains.

A preferred aspect of embodiments (1) and (2) is the performance of the process as a one-step process and/or by selective substrate adhesion to the tenascin-R probe. Thus, single cell suspensions obtained by the enzymatic treatment of the desired central-nervous tissue are sown onto plastic surfaces coated with TN-R proteins or protein fragments. After incubation, preferably for 8-20 hours and preferably in a serum-free medium (which prevents the proliferation of astrocytes and microglial cells), pure oligodendrocyte populations are found on the immobilized TN-R substrates. Other cell types are present in the cell culture supernatant and can be removed completely by changing the medium. Since only one selection step occurs and the selection phase is short as compared to the duration of the previously usual cultivation for several weeks of mixed glial cultures and other selection methods, the resulting cell yield is high. The reason for this is, inter alia, the fact that all oligodendrocytes (of different differentiation stages) from a primary tissue can be selected on TN-R substrates. In contrast, the isolation of oligodendrocytes from mixed glial cultures is accompanied by a high loss of cells (e.g., when the oligodendrocytes and microglia adhering to an astrocyte monolayer are shaken off and microglial cells are further selected).

The isolation process according to the invention according to (1) and (2) allows for the isolation of complete oligodendrocyte populations and is thus also advantageous over immunological selection methods, such as FACS, biomagnetic cell sorting or antibody panning. These only allow for the enrichment of distinct oligodendrocyte populations; oligodendroglial cells not recognized by the antibodies are lost. In particular, the probe used in the process according to (1) and (2) has the property of supporting the survival and transdifferentiation of adult human mesenchymal stem cells into neuronal or oligodendroglial cells, especially under defined serum-free culturing conditions.

Thus, the process according to the invention of (1) and (2) allows for the isolation of oligodendrocyte populations that are often sufficient for further experiments from a single vertebrate, especially from a single rodent. This is of advantage, in particular, if particular effects on mice are examined that occur, for example, in transgenic mice, knockout mice or mice treated with test substances.

The primary tissues used as the starting material of embodiments (1) and (2) can originate from different CNS regions and from different developmental stages. Preferred CNS regions are the brain and individual brain regions (especially forebrain, hindbrain, hippocampus, brain stem), the optical nerve and the spinal chord. The suitable developmental stages include embryonic, fetal, early/late postnatal and adult tissues, preferably early postnatal and adult tissues.

If a single cell suspension is used in the process according to (1) to (2), it contains cells of one or more differentiation stages, preferably a single differentiation stage.

The neural primary tissues used for performing the process according to (1) and (2) originate from lower and higher vertebrates including fish, amphibians, reptiles, birds and mammals, more preferably shark, carp, chicken, rodents including mouse and rat, cattle, pig and human, even more preferably rodents and humans.

The TN-R probe for use in (1) and (2) preferably originates from TN-R of higher and lower vertebrates. Preferably, it is the native TN-R or a fragment of native TN-R.

The recovery of single cell suspensions from primary tissue for use in processes according to embodiment (1) or (2) is effected by methods usual in the art. Thus, the tissue can be converted to tissue fragments and/or single cells in one or more steps mechanically and/or enzymatically. Suitable methods are described in “Zellund Gewebekultur” (T. Lindl, Spektrum Akademischer Verlag, 2002) and “Current Protocols in Neuroscience” (John Wiley & Sons, Inc., 2004, Ed. J. Crawley et al.). The thus obtained cells are resuspended in serum-free medium and then contacted with the TN-R probe. After an incubation time which is sufficient for the complete adsorption of the selected cells (1-48 hours, preferably 8-20 hours) under suitable conditions, the non-adherent cells are removed. For further use, the adhered cells may either remain adherent or be detached from the adsorbent enzymatically, preferably by treatment with trypsin or trypsin-EDTA, collagenase, dispase, pronase, Accutase® or other suitable proteinases, especially with Accutase®. Their further use comprises culturing, also on other substrates or plastic surfaces or in other defined media, for obtaining, for example, immature precursor oligodendrocytes or myelin-competent oligodendrocytes.

The process according to (1) and (2) can be employed independently of whether the TN-R probe and the neural primary tissue originate from organisms of the same species. Thus, isolation of oligodendrocytes over species boundaries is possible. TN-R from different vertebrates can be used for the selection of oligodendrocytes from single cell cultures of other species. Inter alia, this includes the selection of oligodendrocytes from human, pig, cattle, chicken, mouse, rat, frog and other higher vertebrates for TN-R from a different species (including fish, chicken, mouse, rat, cattle, pig, human etc.).

Further, the process according to (1) and (2) can be employed irrespective of the differentiation stage the selected cells are in (e.g., precursors—immature—mature oligodendrocytes). All cells of a cell type are selected irrespective of its developmental stage.

In one aspect, the selection of defined cell populations from single cell suspensions according to (1) and (2) is effected by isolating the cells bound to the TN-R probe, which are designated for further use. In another aspect, in contrast, it is the supernatant that is freed from these cells by the specific adsorption of cells to the TN-R probe and designated for further use as a defined cell population. In yet another aspect, a modified TN-R or a TN-R fragment which is selective for cells other than the starting protein (native TN-R) is used as the TN-R probe.

In one aspect of embodiment (1) or (2), the tenascin-R probe is coupled to a support material by suitable methods for immobilization. Such immobilization may be effected covalently or noncovalently. Such suitable immobilization methods include adequate coupling techniques which leave the specificity of the tenascin-R probe unchanged, such as the covalent cross-linking of the protein with the support material or the immobilization by interaction with a suitable antibody. Preferably, the coupling is effected noncovalently, through an antibody or by covalent cross-linking.

In a preferred embodiment of this aspect of embodiment (1) or (2), for the isolation of cells from primary tissue of neural origin, support materials, such as cell culture plates, are coated with TN-R or with recombinantly prepared TN-R fragments by incubating the support material, preferably a plastic surface, with a solution of the TN-R probe and subsequently washed. The isolation of ultrapure cell populations from cell suspensions is then effected by selective substrate adhesion. Using TN-R, 100% pure oligodendrocyte preparations could be recovered from early postnatal rodent brains: 2×10⁶ oligodendrocytes from a P0-P2 rodent forebrain, 4-6×10⁶ oligodendrocytes from a P5 rodent forebrain, 2×10⁶ oligodendrocytes from a P7-P8 rodent hindbrain. Similar results are possible with recombinantly prepared tenascin-R fragments.

In another embodiment of this aspect, the TN-R probe is immobilized on a plastic surface. This also enables the selective adhesion and isolation of defined cell populations, especially oligodendrocytes, but not of other neural cells (such as astrocytes, microglia or neurons), from the mixed cell populations used as a starting material.

The immobilization is preferably effected by direct contact of the TN-R probe with the support surface. After a sufficient incubation time (1-4 hours), the unbound protein is washed off. The unoccupied binding sites on the surface are subsequently blocked, for example, by incubation with a BSA-containing blocking buffer. The thus coated surfaces can be kept humid until use or be used after drying.

In embodiment (2), an N-terminal fragment of the native TN-R is used as a preferred TN-R probe. Also, the use of a mixture of more than one TN-R probe for the coating of the support material is possible.

The process according to embodiment (1) to (2) is suitable for the recovery of neural cells, especially of oligodendrocytes, for the growth of differentiated cells, especially neural cells, in neurobiological and cell-physiological examinations, in biological and clinical research and for diagnostic and therapeutic methods in vitro and in vivo, especially for the preparation of a medicament for cell therapy and for the therapy of neurodegenerative diseases. Further, it can be used for the detection of neurodegenerative diseases.

Embodiment (8) relates to antibodies that bind specifically to the N-terminal end of TN-R, especially to the N-terminal end of TN-R in at least two species, preferably two vertebrates.

The cross-activity with different species is caused by the preparation process, namely the fact that a suitable host organism is immunized with tenascin-R from at least two different species, preferably from two different vertebrates, by usual methods and isolated in subsequent screening and purification steps. Suitable host organisms for the immunization include, in particular, non-human mammals, such as rodents (mice, rats etc.), rabbits, guinea pigs, goats etc.

The antibodies according to embodiment (8) are applicable in various immunochemical methods based on the detection and/or binding of TN-R, especially in ELISAs, Western blots, histological and cytological examinations and immunoprecipitations. They are also important in in-vitro assays, since their presence can neutralize the inhibitory effect of the TN-R protein on the neuronal cell adhesion and the axon growth. The antibodies react specifically with TN-R in brain extracts or purified TN-R and show no cross-reactivity with other TN proteins. The latter can be concluded from the fact that no reaction takes place with heart or kidney (in mouse containing TN-W, Scherbich, A. et al., J. Cell. Sci. 117: 571-581 (2004)), neonatal skin or skin fibroblast conditioned medium (containing TN-X, Zweers, M. C. et al., Cell Tissue Res. 319: 279-287 (2005)) and TN-C preparations from mouse brain. In particular, the antibodies are suitable for selectively blocking the TN-R fragment (1) and (2) according to the invention.

The monoclonal antibodies according to embodiment (8) can be prepared by culturing the cell line according to embodiment (9). In particular, the cell lines of embodiment (9) are so-called hybridoma cell lines. These are obtainable, for example, by the immunization of a suitable host organism with TN-R from at least two different species as described above, isolation of splenocytes from the host organism, followed by fusing with suitable primary cells, for example, myeloma cells. Depending on the host organism and on the origin of the primary cells, they are homo- or heterohybridoma cells, the former being preferred.

The antibodies of embodiment (8) of the invention are suitable not only for the immunochemical detection of TN-R, but also for the inhibition of the effect of TN-R in vivo and in vitro.

Due to their interaction with TN-R with respect to neuronal cell adhesion and axon growth, the antibodies according to embodiment (8) can be employed for selectively influencing neural development in vivo and in vitro, for the therapy and prophylaxis of traumatic nerve lesions, and for the preparation of medicaments for selectively influencing neural development and for the therapy and prophylaxis of traumatic nerve lesions. Traumatic nerve lesions are produced, for example, after mechanical damage to nerves. The regeneration of the nerve fibers after such lesions is adversely affected by TN-R (Probstmeier, R. et al., J. Neurosci. Res. 60: 21-36 (2000); Zhang, Y. et al., Mol. Cell. Neurosci. 17: 444-459 (2001); Becker, C. C. et al., Mol. Cell. Neurosci. 26: 376-389 (2004); Xu, G. et al., J. Neurochem. 91: 1018-1023 (2004)).

Therefore, in embodiment (14), the invention also relates to a process for the therapy and prophylaxis of traumatic neural lesions and for the selective influencing of neural development, comprising the step of administering a suitable amount of antibody according to embodiment (8) to a patient in need of such treatment. The amount of antibody administered and the necessary dosage will be determined by the attending physician on a case by case basis. It depends, inter alia, on the age, body weight and constitution of the patient on the one hand and on the kind and severity of the disease to be treated on the other hand.

The kit according to embodiment (10) preferably contains the protein defined in embodiment (3) or a stock culture of the cell line for the production of such protein. More preferably, such a kit contains human tenascin-R or its fragments according to the invention and/or a stock culture of cells which are suitable for the recombinant production of these proteins.

A preferred aspect of embodiment (10) is a kit in which the tenascin-R probe has been bound to a support material by an adequate coupling technique, and/or which further contains TN-R antibodies (for example, for confirming the immobilization efficiency of the TN-R probe), one or more enzymatic solutions for cell dissociation, optionally agents for the detection of the binding of cells to the tenascin-R probe, buffers and/or culture media. The buffers and media include, in particular, blocking buffers and defined serum-free culture media. Antibodies contained in the kit are preferably antibodies of embodiment (8).

In embodiment (11), the use of a TN-R fragment or a TN-R fusion protein as defined in embodiment (3) is preferred.

A preferred use of the TN-R probe according to the invention is the use of the tenascin-R probe for the recovery of oligodendrocytes according to embodiment (11). A related aspect of embodiment (11) is the culturing of differentiated cells from the thus obtained cell populations. The differentiation-promoting effect of the native TN-R has been described (Pesheva, P. et al., J. Neurosci. 17: 4642-51 (1997)).

The diagnostic methods according to embodiment (11) can be performed in vivo and in vitro, but preferably in vitro. For the use according to embodiment (11), a tenascin-R probe according to embodiment (4) is preferably used, especially human tenascin-R or its fragments according to the invention. Said tenascin-R probe may have been purified from sources in which it naturally occurs or may have been prepared recombinantly. Preferably, a recombinant tenascin-R probe is used.

Neurodegenerative diseases associated with a loss of oligodendrocytes (by cell death) or myelin, such as multiple sclerosis (MS), are characterized in that a remyelinization cannot take place in the affected regions, or only so to a small extent. This is mainly due to the fact that the existing “traumatic” (i.e., altered due to the action of pathological stimuli) precursor and immature oligodendrocytes are not capable of differentiating/remyelinizing. The invention offers approaches for novel diagnostic and/or therapeutic methods:

For the diagnosis of MS, the quick (within 1-2 days) selection of “traumatic” cells on TN-R probes is particularly suitable, preferably from an animal model or from biopsy samples from patients. This enables the performance of direct examinations of their molecular profile and/or the development of diagnostic markers. For the development of a medicament for cell therapy, “traumatic” oligodendrocytes can be treated with different candidate drugs in order to determine the influence of the latter on the “recovery of traumatic cells”, or the remyelinization potency of such cells.

The method according to the invention is also suitable for the selection of “normal” adult oligodendrocytes that are selected under traumatic conditions in vitro (by adding relevant cytokines or cerebrospinal fluid samples from patients/sick animals) and subsequently treated with candidate drugs before culturing. Such an in-vitro system allows examinations relating to the traumatic alterations occurring in oligodendrocytes that allow conclusions to be drawn to such alterations in vivo.

Further, such cultured oligodendrocytes can serve for the development of diagnostic markers, wherein different stages of traumatic alterations can also be established. The thus obtained oligodendrocytes are also suitable for the screening for candidate drugs that compensate for cell death and/or a lacking myelinization competence of oligodendrocytes under traumatic conditions, or may be employed as a medicament for the cell therapy in vivo.

Thus, a preferred aspect of embodiment (11) is the use of the tenascin-R probe for the diagnosis of multiple sclerosis (MS) and for the preparation of a medicament for MS.

Thus, another preferred aspect of embodiment (11) is the use of the tenascin-R probe for the preparation of a medicament for cell therapy and for the therapy of neurodegenerative diseases accompanied by a loss of oligodendrocytes or myelin, especially multiple sclerosis and periventricular leukomalacia (PVL).

Embodiment (13) relates to a process for cell therapy and for the therapy of neurodegenerative diseases accompanied by a loss of oligodendrocytes or myelin, especially multiple sclerosis and periventricular leukomalacia (PVL), comprising the step of administering a pharmacologically sufficient amount of the TN-R probe to a patient in need of such treatment. The amount administered and the necessary dosage will be determined by the attending physician on a case by case basis. It depends, inter alia, on the age, body weight and constitution of the patient on the one hand and on the kind and severity of the disease to be treated on the other hand.

The process (12) for preparing oligodendrocytes from isolated stem cells is preferably performed with neural or non-neural stem cells. Particularly preferred isolated stem cells are progenitor cells of neural or hematopoietic origin. Thus, human neural stem cells can be selectively differentiated into mature oligodendrocytes in the presence of TN-R.

Preferably, the process (12) is performed with TN-R from higher vertebrates, more preferably with a TN-R probe according to the invention, even more preferably with native TN-R or a TN-R fragment or fusion protein of embodiment (4).

The hybridoma cell lines to-R4 (producer of antibody R4) and to-R6 (producer of antibody R6) were deposited on Dec. 2, 2005, under the accession Nos. DSM ACC2754 and DSM ACC2753, respectively, with the DSMZ, Deutsche Sammlung von Mikroorganismen and Zellkulturen GmbH, Mascheroder Weg 1b, 38124 Braunschweig, Germany.

The invention is further illustrated by means of the following Examples. However, they do not limit the scope of protection of the invention.

### Examples

Solutions/Media Employed:

### 1. HBSS (Hank's Balanced Salt Solution) (Sigma)

2. Enzymatic solutions for cell dissociation: 1% (w/v) trypsin solution: 1% (w/v) trypsin (cell culture tested), 0.1% (w/v) DNase I, 1 mM EDTA, 0.8 mM MgSO₄, 10 mM HEPES in HBSS (Ca/Mg-free). DNase solution: 0.05% (w/v) DNase I, 10 mM HEPES in BME (Basal Medium Eagle).
3. Support medium: DMEM (Sigma) supplemented with 10% (v/v) FCS (fetal calf serum), penicillin (100 units/ml) and streptomycin (0.1 mg/ml).
4. Defined serum-free growth medium: DMEM (Sigma) supplemented with insulin (10 μg/ml), progesterone (0.06 μg/ml), putrescine (16 μg/ml), sodium selenite (0.22 μM), transferrin (0.1 mg/ml), HEPES (25 mM), gentamicin (25 μg/ml), penicillin (100 units/ml) and streptomycin (0.1 mg/ml), and the growth factors FGF-B (human recombinant basic FGF, Peprotech: 50 ng/ml) and PDGF-BB (human recombinant PDGF, Peprotech: 10 ng/ml) (which support the proliferation of oligodendrocyte progenitor cells).
5. Serum-free glial differentiation medium: serum-free growth medium without growth factors (FGF-B and PDGF-BB).
6. Trypsin/EDTA solution for hMSC (Lonza).
7. MSCGM (mesenchymal stem cell growth medium) for hMSC (Lonza).
8. Accutase® solution (Sigma)
9. Defined serum-free neuromedium: DMEM/Ham's F-12 (Sigma, with L-glutamine and 15 mM HEPES) supplemented with EGF (human recombinant epidermal growth factor, 20 ng/ml) and FGF-B (basic fibroblast growth factor, 20 ng/ml).
10. Serum-free neuronal differentiation medium: neurobasal medium (Invitrogen) supplemented with 1% Glutamax (Invitrogen), 1% N2 supplement (Invitrogen), B-27 supplement 1:50 (Invitrogen), gentamicin (25 μg/ml), penicillin (100 units/ml) and streptomycin (0,1 mg/ml), and the growth factor BDNF (human recombinant brain-derived neurotrophic factor, 10 ng/ml).

Antibodies Employed

The R4 and R6 antibodies are obtainable from the hybridoma cell lines tn-R4 and tn-R6, deposited on Dec. 2, 2005, under the accession Nos. DSM ACC2754 and DSM ACC2753, respectively, with the DSMZ (Deutsche Sammlung von Mikroorganismen and Zellkulturen GmbH) (see also WO 2006/067094). V-5 antibodies (Sigma, Cat. No. V8012), O4 antibodies (Sigma, Cat. No. O7139).

### Example 1: Preparation of a Human TN-R-Cys (H-TN-R-Cys) Fragment

For the preparation of a recombinant eukaryotically expressed H-TN-R-Cys fragment, the following sequence region was selected:

H-TN-R-Cys: by 151-648 (of SEQ ID No. 1, cysteine-rich region)
Product length: 498 bp

For the recovery of human TN-R-specific mRNA, the human neuroblastoma cell line SH-SY5Y is used (Woodworth et al., 2004). Total RNA is purified from these cells using TRIzol reagent (Invitrogen). The cDNA synthesis from these RNA preparations is effected using “random hexamer” primers by means of the SuperScript II system (Invitrogen). For the preparation of H-TN-R-Cys-specific cDNA fragment, the following primers were used:

H-TN-R-Cys-UP (SEQ ID No. 3) bp 151-173:
TCCATGATCAAGCCTTCAGAGTG
H-TN-R-Cys-DO (SEQ ID No. 4) bp 627-648:
GATGCAGCCACAGGACTCAAAG

The PCR fragment obtained is cloned into the pcsecTag/FRT/V5-His-TOPO vector (Invitrogen) by means of the TOPO/TA cloning system (Invitrogen), which makes use of the overhanging A residues of the PCR products when Taq polymerase is used. After stable integration into eukaryotic Flp-In cell lines (see below), this vector allowed for the secretion of the desired protein fragment provided with a 6×His peptide at the C terminus into the cell culture supernatant. PolyHis-bearing fragments were purified by nickel chelate chromatography from collected cell culture supernatants.

Flp-In 293 cells (Invitrogen), which are derived from the human kidney cell line HEK 293, were used as producers of the H-TN-R-Cys fragment. In Flp-In cells, the plasmid pFRT/lacZEo (Invitrogen) is stably integrated. This vector contains an FRT region which is specifically recognized by Flp recombinase. When the Flp 293 cells are simultaneously transfected with the pOG44 plasmid, which enables the expression of Flp recombinase, and the pcsecTag/FRT/V5-His-TOPO vector, which bears the base sequence of the H-TN-R-Cys fragment, incorporation of the pcsecTag/FRT/V5-His-TOPO vector occurs at the FRT region.

For bacterial expression, the Cys fragment can be cloned into a prokaryotic expression vector, e.g., the vector pIN-III-ompA2 (SEQ ID No. 5; address PubMed nucleotides: NM_{—}013045). The foreign gene obtained by cell-free synthesis (Cys region+His tag) has flanking EcoRI- and BamHI-specific restriction sites, which enable the incorporation of the foreign gene into the EcoRI and BamHI cloning sites of the pIN-III-ompA2 vector after a corresponding digestion with restriction enzymes.

The expression of the protein is effected in E. coli strain JA221 after vector transformation. The selection of transformants is effected by culturing in ampicillin-containing culture media. The protein is secreted into the periplasmic space of the E. coli cell, which is disrupted by osmotic shock, and the protein is purified by affinity chromatography through a C-terminal His tag.

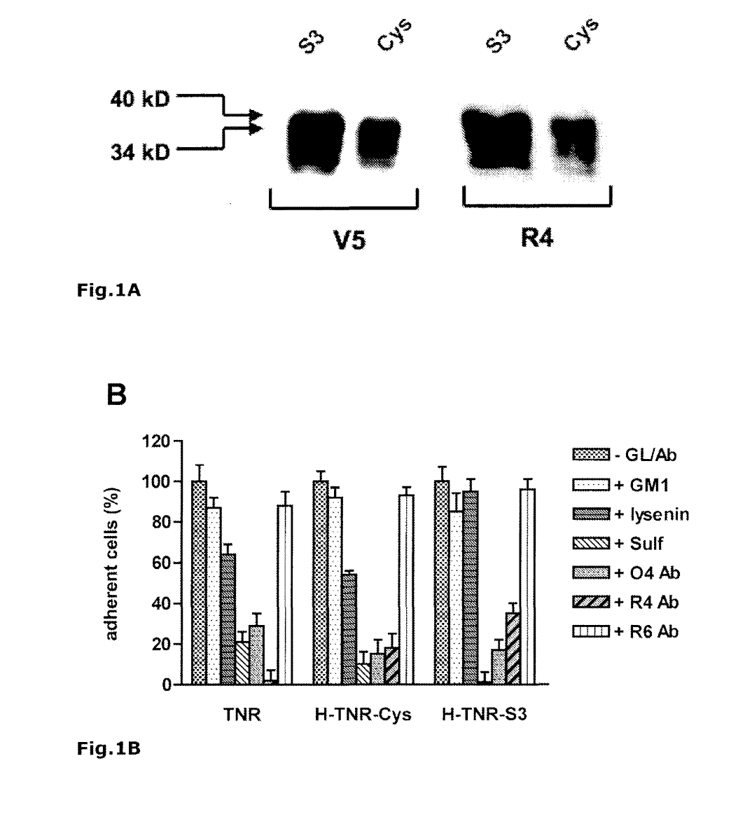

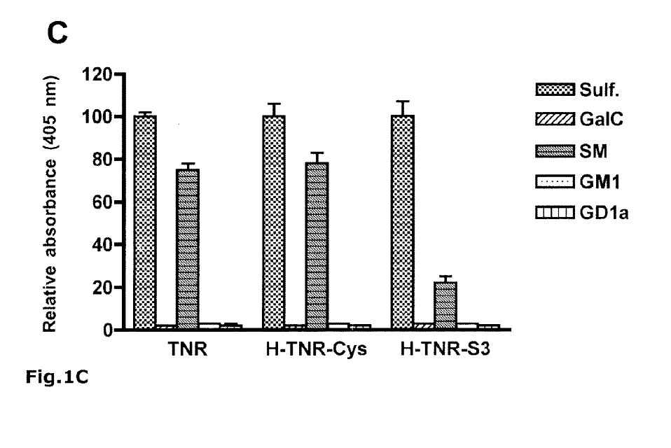

The H-TN-R-Cys fragment prepared by the Flp-In expression system shows a molecular weight of 34 kD in SDS-PAGE and is recognized by tag-specific (V5) and TN-R-specific (R4) monoclonal antibodies in Western blot analyses (FIG. 1A). Western blot and functional analyses of recombinant H-TN-R-Cys and H-TN-R-S1 to −5 fragments (S1=24 to 328, S2=324 to 1134, S3=1120 to 1358, S4=24 to 506, S5=498 to 955, S6=949 to 1358 of SEQ ID No. 2, see also WO 06/067094) demonstrate that the R4 antibody (1) recognizes an epitope in the Cys region (H-TN-R-Cys fragment) and the FNG domain (H-TN-R-S3 fragment) of TN-R (FIG. 1A), (2) blocks the sulfatide-mediated cell adhesion to TN-R/TN-R fragments (FIG. 1B) and binds to the sulfatide binding sites of TN-R, or that H-TN-R-Cys and H-TN-R-53 fragments bind sulfatides (FIG. 1C).

### Example 2: Selective Purification of Oligodendrocytes from CNS Tissue of Mammals Using Recombinant H-TN-R Fragments

As the starting material, postnatal mouse brains (either the total brain or isolated forebrain regions or preparations of the optical nerve) of the age stages postnatal day 1 (P1) to 4 (P4) were used. After mechanical comminution in accordance with origin and age, isolated brain regions were treated with 0.5 to 1% (w/v) trypsin solution (P1 to P2 brains: with 0.5% (w/v) trypsin solution for 10 min at room temperature (RT), P4 brains: with 0.5% (w/v) trypsin solution for 15 min at RT. After a substantial volume of HBSS was added, the tissue portions were pelletized at 600 g for 5 minutes at 4° C.

For the recovery of single cells, the pelletized tissue pieces were taken up in DNase solution and pipetted up and down repeatedly in a Pasteur pipette having a narrowed tip diameter. The coarse cell suspension obtained was diluted in a five to 10 fold volume of support medium and incubated on ice for 5 minutes. The supernatant containing the single cell suspension was centrifuged at 600 g for 5 min at 4° C., and the pelletized single cells were resuspended in serum-free medium. The thus obtained single cell suspensions contained all cell types present in the corresponding brains/brain regions (neurons, astrocytes, oligodendrocytes, microglia, meningial and endothelial cells).

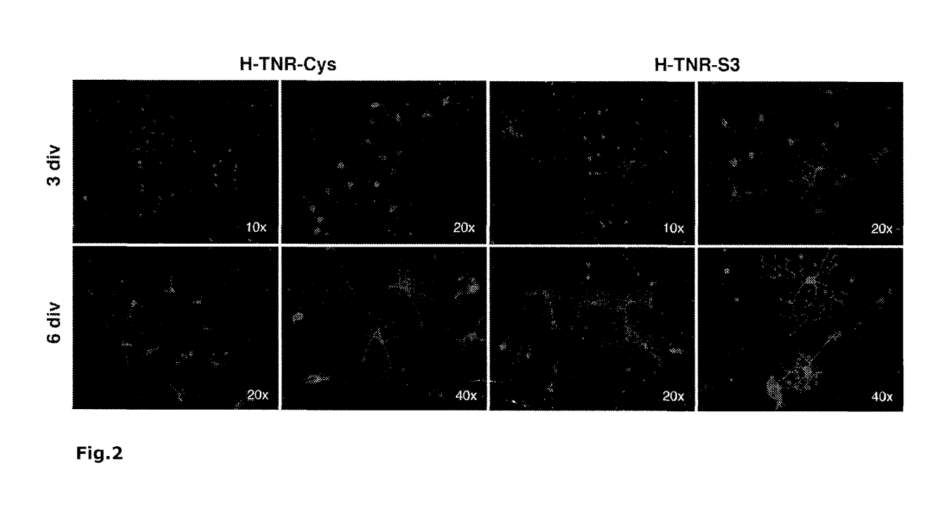

For the recovery of pure oligodendrocyte populations, the single cell suspensions presented in the preceding paragraph (1-2×10⁶ cells/ml in serum-free medium) were plated onto cell culture plates coated with H-TN-R fragments (see below) and cultured in a CO₂ incubator (5% CO₂) for 8-20 hours. Non-adherent cells were washed away with HBSS, and adherent cells were further cultured in serum-free medium. Subsequently, the cells adherent on H-TN-R fragments were incubated with a sulfatide-specific monoclonal mouse antibody (O4; Bansai, R. et al., J. Neurosci. Res. 24: 548-557 (1989)) (30 min at RT). After fixing the cells with 4% (v/v) formaldehyde in PBS for 10 min at RT, the binding of the O4 antibody onto the cells was detected by incubation with cyanine-3- or FITC-coupled anti-mouse Ig antibodies (20 min at RT) by fluorescence microscopy. On the substrate, there remained 99±1% of O4-stainable cells, i.e., only oligodendrocytes (FIG. 2).

The cells obtained by this one-step process could be detached from the substrate for further intended uses by treatment with Accutase® (Sigma) and cultured further on other substrates/plastic surfaces or in other defined media for the recovery of, for example, immature or mature, myelin-competent oligodendrocytes.

For the preparation of substrates of H-TN-R fragments, plastic surfaces (cell culture dishes, plates etc.) were incubated with H-TN-R-Cys or H-TN-R-S3 fragments (10 μg/ml in 0.1 M NaHCO₃) for 2 hours at RT, washed with PBS (2-3 times) and subsequently used as a substrate for oligodendrocyte selection. The substrates prepared from H-TN-R fragments may also be dried and stored sterilely at −20° C. after coating, without losing the specific adhesive properties of the fragments for oligodendrocytes.

### Example 3: Differentiation Process for Glial Cells from Adult Human Mesenchymal Stem Cells (hMSC) by Means of Recombinant H-TN-R Fragments

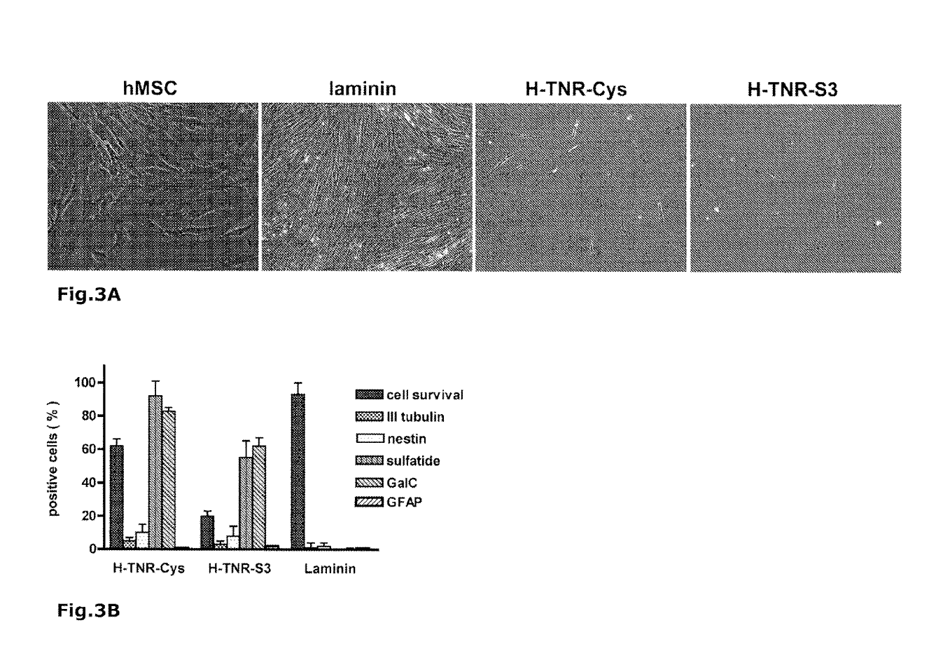

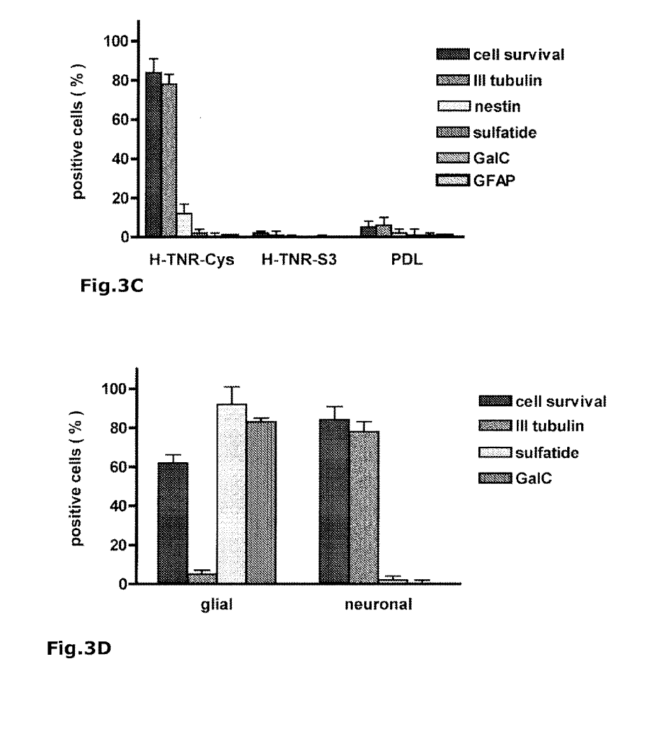

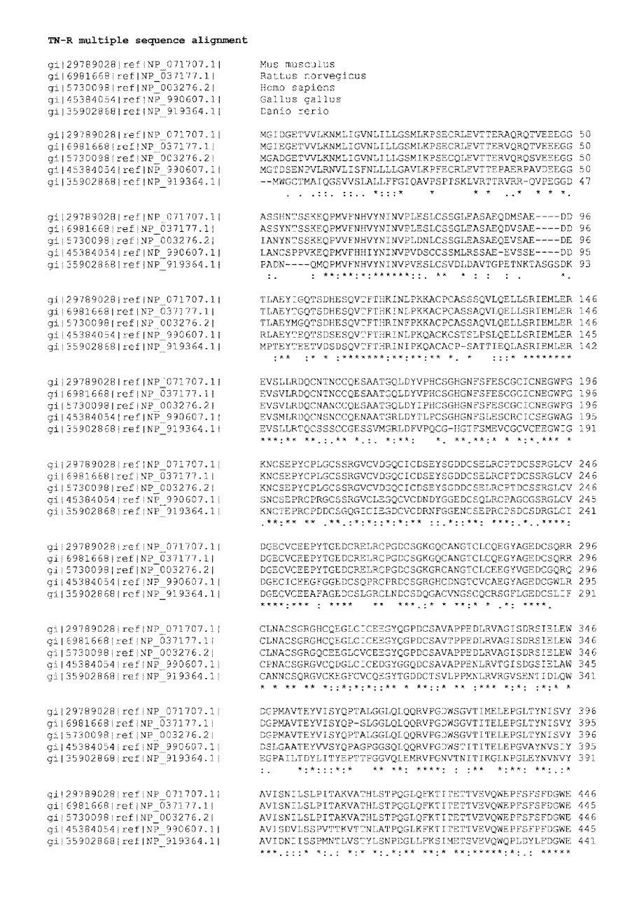

Adult human mesenchymal stem cells (hMSC) from bone marrow (Lonza; CD105+, CD166+, CD29+ and CD44+) were used as starting materials. The cells, which had been isolated from normal human bone marrow, were proliferated in MSCGM for 3 to 4 passages. For the recovery of single cells, the cells growing as a monolayer were dissociated with trypsin/EDTA solution, and after the addition of a larger volume of MSCGM, the single cell suspension was pelletized at 600 g for 5 minutes at 4° C., and the pelletized single cells were resuspended in serum-free growth medium. For the recovery of glial cell populations, single cell suspensions (1-2×10⁶ cells/ml) were plated onto uncoated dishes and cultured in serum-free growth medium in a CO₂ incubator (5% CO₂) for 6 to 9 days. The cell culture medium was replaced every 2-3 days. Subsequently, adherent cells were trypsinized, single cell suspensions (1×10⁶ cells/ml) were plated onto dishes coated with laminin (control substrate) or H-NTR fragments (as described above), and cultured for another 2 to 5 days in serum-free growth medium (FIG. 3A). Then, the growth medium was replaced by glial differentiation medium, and the cells were further cultured therein for 3 days. Subsequently, the cells adherent to the substrates were fixed with 4% (v/v) formaldehyde and 5% (w/v) sucrose in PBS for 30 min at RT, and stained by indirect immunofluorescence using neuron-(βIII tubulin) and glia-specific antibodies (against nestin, sulfatide, galactocerebroside (GalC) and GFAP) for 30 min at RT (for sulfatide and GalC). For the detection of βIII tubulin, nestin and GFAP, the fixed cells were first permeabilized with a 0.25% Triton X-100 solution in PBS (20 min at RT) and then incubated with molecule-specific antibodies (abcam) over night at 4° C. The binding of the corresponding antibodies was visualized by fluorescence microscopy upon incubation (30 min at RT) with cyanine-3- or ALEXA-488-coupled secondary antibodies. On the H-TN-R-S3 and H-TN-R-Cys substrates, 60% (on H-TN-R-S3) to 90% (on H-TN-R-Cys) of sulfatide/GalC-positive cells, i.e., predominantly oligodendrocytes, were detected, wherein the survival rate of oligodendroglial-differentiated cells on H-TN-R-Cys (60% of the initial cell count) was three times the rate on H-TN-R-S3 (FIG. 3B, 3D).

### Example 4: Differentiation Process for Neuronal Cells from Adult Human Mesenchymal Stem Cells (hMSC) by Means of Recombinant H-TN-R Fragments

Adult human mesenchymal stem cells (hMSC) from bone marrow (Lonza; CD105+, CD166+, CD29+ and CD44+) were used as starting materials. The cells, which had been isolated from normal human bone marrow, were proliferated in MSCGM for 3 passages. Cells from monolayers were dissociated with trypsin/EDTA solution, and after the addition of a larger volume of MSCGM, the single cell suspension was pelletized at 600 g for 5 minutes at 4° C., and the pelletized single cells were resuspended in serum-free neuromedium. For the recovery of neuronal cell populations, single cell suspensions (1-2×10⁵ cells/cm²) were plated onto dishes coated with 0.8% (w/v) agarose type VII (Sigma) (5 ml/95 mm dish), and cultured for 3 to 5 days in neuromedium in a CO₂ incubator (5% CO₂) until neurospheres had formed. The cell culture medium was replaced every 2-3 days. Subsequently, neurospheres were pelletized by centrifugation, dissociated with Accutase® (Sigma) for 15 min at RT, and resuspended in neuronal differentiation medium. Single cell suspensions (0.5×10⁶ cells/ml) were plated onto dishes coated with laminin or poly-D-lysine (PDL, control substrates) and H-TNR fragments (as described above), and cultured for 5 to 10 days in neuronal differentiation medium. The cell culture medium was replaced every 2-3 days. Subsequently, the cells adherent to the substrates were fixed with 4% (v/v) formaldehyde in PBS for 30 min at RT, and analyzed by indirect immunofluorescence using neuron- and glia-specific antibodies (as described above). While almost no stem cells survived in the neuronal differentiation medium on H-TN-R-S3 substrates, 80% of the cells with long axon-like protrusions could be identified on H-TN-R-Cys substrates as βIII-tubulin-positive and thus as neurons/neuronal progenitors. The survival rate of transdifferentiated cells was more than 80% of the initial cell count derived from neurospheres (FIGS. 3C, 3D).

1. A process for the isolation and purification of neural cells from neural primary tissue of vertebrates, comprising selecting the cells from a single cell suspension by means of a probe containing tenascin-R (“tenascin-R probe”) selected from (a) the N-terminal fragment of native tenascin-R (TN-R) corresponding to the human tenascin-R fragment encoded by nucleotides 151-648 of SEQ ID No. 1, (b) homologues and fragments of (a), and (c) fusion proteins comprising a domain with (a) or (b).

2. The process according to claim 1, wherein said N-terminal tenascin-R fragment (a) is a tenascin-R fragment which is a fragment with amino acid residues 24 to 189 of SEQ ID No. 2.

3. The process according to claim 1, wherein said fragment (b) is
(i) a fragment of (a) in which up to 10, amino acid residues have been cleaved off from the N and/or C terminus, and/or which has at least 150 amino acid residues; and/or
(ii) a substitution, deletion and/or addition mutant of (a) or (b).

4. The process according to claim 1, wherein said tenascin-R fusion protein (c)
(i) has at least one domain with a tenascin-R fragment (a) or (b) and a functional domain including one or more other functional peptides or proteins, or
(ii) is composed of two or more, functional tenascin-R fragments (a) or (b).

5. The process according to claim 1, wherein
(i) said TN-R fragment originates from vertebrates; and/or
(ii) said tenascin-R probe contains further functional peptide or protein sequences and/or is coupled to a support; and/or
(iii) said tenascin-R fragment is a peptide having the sequence of amino acid residues 24 to 189 of SEQ ID No. 2.

6. The process according to claim 1, wherein
(i) said process is adapted for the isolation and purification of glial cells; and/or
(ii) said process is adapted for the transdifferentiation of adult human mesenchymal stem cells into neuronal or oligodendroglial cells; and/or
(ii) said vertebrate primary tissue originates from lower and higher vertebrates; and/or
(iii) the isolation of the cells is effected by selective substrate adhesion to the tenascin-R probe and/or in a single purification step.

7. The process according to claim 1, wherein said single cell suspension
(i) is prepared from embryonic, fetal, early or late postnatal and/or adult tissues; and/or
(ii) is prepared from tissue from different regions of the nerve system; and/or
(iii) contains cells of one or more differentiation stages.

8. The process according to claim 1, wherein
(i) said tenascin-R probe is bound to a support material by non-covalent interactions or by another adequate coupling technique which does not change the specificity of the tenascin-R probe; and/or
(ii) said single cell suspension is contacted with said tenascin-R probe so that tenascin-R-binding cells present in said single cell suspension become bound to said probe; and/or
(iii) isolation of these cells from the cell culture is effected by specific binding of neural stem cells from said single cell suspension to said tenascin-R probe, the unbound cells are removed, and optionally the cells bound to the support material through said tenascin-R probe are subsequently detached from the support material by trypsinization, incubation with a solution of proteolytic and collagenolytic enzymes (ACCUTASE®) or another adequate method; and/or
(iv) the bound cells are detected by immunological methods; and/or
(v) the process is effected in vitro.

9. The process according to claim 1, which is adapted
(i) for obtaining neural cells, for growing differentiated cells, in neurobiological and cell-physiological examinations, in biological and clinical research and for diagnostic and therapeutic processes in vitro and in vivo; and/or
(ii) for the detection of neurodegenerative diseases.

10. A tenascin-R fragment or tenascin-R fusion protein as defined in claim 1.

11. A DNA which codes for a tenascin-R fragment or tenascin-R fusion protein according to claim 10.

12. A vector which comprises a DNA according to claim 11.

13. A host organism transformed/transfected with a vector according to claim 12.

14. A process for preparing a tenascin-R fragment or TN-R fusion protein, comprising the step of culturing said host organism according to claim 13.

15. An antibody obtainable by the immunization of a suitable host organism with a tenascin-R fragment according to claim 10.

16. The antibody according to claim 15, which
(i) is specific for a tenascin-R fragment; and/or
(ii) is obtainable by immunization with tenascin-R fragments from at least two different species, and/or which binds to the N-terminal tenascin-R of at least two different species; and/or
(iii) is monoclonal.

17. A cell line or hybridoma cell line which produces a monoclonal antibody according to claim 15.

18. Method of using the antibody according to claim 15
(i) for the immunochemical detection of TN-R;
(ii) for inhibiting the effect of TN-R;
(iii) for influencing the neural development; and
(iv) for preparing medicaments for the therapy and prophylaxis of traumatic nerve lesions and medicaments for selectively influencing the neural development.

19. A kit for the isolation and purification of neural cells, according to the process according to claim 1, especially containing
(i) a tenascin-R probe; and/or
(ii) a vector which codes for the tenascin-R probe defined in (i); and/or
(iii) a stock culture of a cell line which is adapted for expressing said tenascin-R probe.

20. The kit according to claim 19, wherein
(i) said probe is bound to a support material; and/or
(ii) the kit further comprises tenascin-R antibodies; and/or
(iii) the kit further comprises enzymatic solutions for cell dissociation, buffers and/or culture media.

21. Method of using a tenascin-R fragment or tenascin-R fusion protein as defined in claim 10 for obtaining neural cells, for growing differentiated cells, in neurobiological and cell-physiological examinations, in biological and clinical research and for diagnostic and therapeutic processes in vitro and in vivo.

22. A process for preparing oligodendrocytes from isolated stem cells in vitro by incubating the stem cells in the presence of a tenascin-R fragment or tenascin-R fusion protein as defined in claim 10.

23. The process according to claim 22, wherein said isolated stem cells are neural or non-neural stem cells which have the potential for sulfatide expression.

24. A process for cell therapy or for the therapy of neurodegenerative diseases accompanied by a loss of oligodendrocytes or myelin, comprising the step of administering a tenascin-R fragment or tenascin-R fusion protein as defined in claim 10 to a human or animal patient.

25. A process for the therapy and prophylaxis of traumatic nerve lesions or for selectively influencing the neural development, comprising the step of administering a pharmacologically sufficient amount of the antibody according to claim 15 to a human or animal patient in need of such treatment.


SEQUENCE LISTING (10 sequences)

SEQ ID NO: 1 - DNA, 4716 bp, Homo sapiens
  CDS (82)..(4158)
  sig_peptide (82)..(150)
ccttggtttccgttgcagattcccacaactccatgctgtgtgctgcaggctggtcctgaacccagatctctggctgagaggatgggggcagatggggaaacagtggttctgaagaacatgctcattggcgtcaacctgatccttctgggctccatgatcaagccttcagagtgtcagctggaggtcaccacagaaagggtccagagacagtcagtggaggaggagggaggcattgccaactacaacacgtccagcaaagagcagcctgtggtcttcaaccacgtgtacaacattaacgtgcccttggacaacctctgctcctcagggctagaggcctctgctgagcaggaggtgagtgcagaagacgagactctggcagagtacatgggccagacctcagaccacgagagccaggtcacctttacacacaggatcaacttccccaaaaaggcctgtccatgtgccagttcagcccaggtgctgcaggagctgctgagccggatcgagatgctggagagggaggtgtcggtgctgcgagaccagtgcaacgccaactgctgccaagaaagtgctgccacaggacaactggactatatccctcactgcagtggccacggcaactttagctttgagtcctgtggctgcatctgcaacgaaggctggtttggcaagaattgctcggagccctactgcccgctgggttgctccagccggggggtgtgtgtggatggccagtgcatctgtgacagcgaatacagcggggatgactgttccgaactccggtgcccaacagactgcagctcccgggggctctgcgtggacggggagtgtgtctgtgaagagccctacactggcgaggactgcagggaactgaggtgccctggggactgttcggggaaggggagatgtgccaacggtacctgtttatgcgaggagggctacgttggtgaggactgcggccagcggcagtgtctgaatgcctgcagtgggcgaggacaatgtgaggaggggctctgcgtctgtgaagagggctaccagggccctgactgctcagcagttgcccctccagaggacttgcgagtggctggtatcagcgacaggtccattgagctggaatgggacgggccgatggcagtgacggaatatgtgatctcttaccagccgacggccctggggggcctccagctccagcagcgggtgcctggagattggagtggtgtcaccatcacggagctggagccaggtctcacctacaacatcagcgtctacgctgtcattagcaacatcctcagccttcccatcactgccaaggtggccacccatctctccactcctcaagggctacaatttaagacgatcacagagaccaccgtggaggtgcagtgggagcccttctcattttccttcgatgggtgggaaatcagcttcattccaaagaacaatgaagggggagtgattgctcaggtccccagcgatgttacgtcctttaaccagacaggactaaagcctggggaggaatacattgtcaatgtggtggctctgaaagaacaggcccgcagcccccctacctcggccagcgtctccacagtcattgacggccccacgcagatcctggttcgcgatgtctcggacaccgtggcttttgtggagtggattccccctcgagccaaagtcgatttcattcttttgaaatatggcctggtgggcggggaaggtgggaggaccaccttccggctgcagcctcccctgagccaatactcagtgcaggccctgcggcctggctcccgatacgaggtgtcagtcagtgccgtccgagggaccaacgagagcgattctgccaccactcagttcacaacagagatcgatgcccccaagaacttgcgagttggttctcgcacagcaaccagccttgacctcgagtgggataacagtgaagccgaagttcaggagtacaaggttgtgtacagcaccctggcgggtgagcaatatcatgaggtactggtccccaggggcattggtccaaccaccagggccaccctgacagatctggtacctggcactgagtatggagttggaatatctgccgtcatgaactcacagcaaagcgtgccagccaccatgaatgccaggactgaacttgacagtccccgagacctcatggtgacagcctcctcggagacctccatctccctcatctggaccaaggccagtggccccattgaccactaccgaattacctttaccccatcctctgggattgcctcagaagtcaccgtacccaaggacaggacctcatacacactaacagatctagagcctggggcagagtacatcatttccgtcactgctgagaggggtcggcagcagagcttggagtccactgtggatgctttcacaggcttccgtcccatctctcatctgcacttttctcatgtgacctcctccagtgtgaacatcacttggagtgatccatctcccccagcagacagactcattcttaactacagccccagggatgaggaggaagagatgatggaggtctccctggatgccaccaagaggcatgctgtcctgatgggcctgcaaccagccacagagtatattgtgaaccttgtggctgtccatggcacagtgacctctgagcccattgtgggctccatcaccacaggaattgatcccccaaaagacatcacaattagcaatgtgaccaaggactcagtgatggtctcctggagccctcctgttgcatctttcgattactaccgagtatcatatcgacccacccaagtgggacgactagacagctcagtggtgcccaacactgtgacagaattcaccatcaccagactgaacccagctaccgaatacgaaatcagcctcaacagcgtgcggggcagggaggaaagcgagcgcatctgtactcttgtgcacacagccatggacaaccctgtggatctgattgctaccaatatcactccaacagaagccctgctgcagtggaaggcaccagtgggtgaggtggagaactacgtcattgttcttacacactttgcagtcgctggagagaccatccttgttgacggagtcagtgaggaatttcggcttgttgacctgcttcctagcacccactatactgccaccatgtatgccaccaatggacctctcaccagtggcaccatcagcaccaacttttctactctcctggaccctccggcaaacctgacagccagtgaagtcaccagacaaagtgccctgatctcctggcagcctcccagggcagagattgaaaattatgtcttgacctacaaatccaccgacggaagccgcaaggagctgattgtggatgcagaagacacctggattcgactggagggcctgttggagaacacagactacacggtgctcctgcaggcagcacaggacaccacgtggagcagcatcacctccaccgctttcaccacaggaggccgggtgttccctcatccccaagactgtgcccagcatttgatgaatggagacactttgagtggggtttaccccatcttcctcaatggggagctgagccagaaattacaagtgtactgtgatatgaccaccgacgggggcggctggattgtattccagaggcggcagaatggccaaactgattttttccggaaatgggctgattaccgtgttggcttcgggaacgtggaggatgagttctggctggggctggacaatatacacaggatcacatcccagggccgctatgagctgcgcgtggacatgcgggatggccaggaggccgccttcgcctcctacgacaggttctctgtcgaggacagcagaaacctgtacaaactccgcataggaagctacaacggcactgcgggggactccctcagctatcatcaaggacgccctttctccacagaggatagagacaatgatgttgcagtgactaactgtgccatgtcgtacaagggagcatggtggtataagaactgccaccggaccaacctcaatgggaagtacggggagtccaggcacagtcagggcatcaactggtaccattggaaaggccatgagttctccatcccctttgtggaaatgaagatgcgcccctacaaccaccgtctcatggcagggagaaaacggcagtccttacagttctgagcagtgggcggctgcaagccaaccaatattttctgtcatttgtttgtattttataatatgaaacaaggggggagggtaatagcaatgtgttttgcaacatattaagagtatgtgaaggaagcagggatgtcgcaggaatccgctggctaacatctgctcttggtttctgctgccctggagcctgaccctcagtctccattctccctcctacccaggcctcctcaaccttcacctcctttcccaccaaggaggagaagtaggaagttttcttaaagggccaattcaaagccaagtcgtggggtgcagattgttatggtgacaggcacacacatttttctacccttcttctgagatgtcctctgccttccaggtatttgtgattttgtcacagcctgacatggccaggttctcacactggcccagagaaaagagcctcagcaagagagttttgccaacaattccccttaaaaggaaacagatcaactacaccgcatcccaacaacccaggttcttttccttccttccttccttcctcccttccttctttcctgccttccc

SEQ ID NO: 2 - protein, 1358 aa, Homo sapiens
MGADGETVVLKNMLIGVNLILLGSMIKPSECQLEVTTERVQRQSVEEEGGIANYNTSSKEQPVVFNHVYNINVPLDNLCSSGLEASAEQEVSAEDETLAEYMGQTSDHESQVTFTHRINFPKKACPCASSAQVLQELLSRIEMLEREVSVLRDQCNANCCQESAATGQLDYIPHCSGHGNFSFESCGCICNEGWFGKNCSEPYCPLGCSSRGVCVDGQCICDSEYSGDDCSELRCPTDCSSRGLCVDGECVCEEPYTGEDCRELRCPGDCSGKGRCANGTCLCEEGYVGEDCGQRQCLNACSGRGQCEEGLCVCEEGYQGPDCSAVAPPEDLRVAGISDRSIELEWDGPMAVTEYVISYQPTALGGLQLQQRVPGDWSGVTITELEPGLTYNISVYAVISNILSLPITAKVATHLSTPQGLQFKTITETTVEVQWEPFSFSFDGWEISFIPKNNEGGVIAQVPSDVTSFNQTGLKPGEEYIVNVVALKEQARSPPTSASVSTVIDGPTQILVRDVSDTVAFVEWIPPRAKVDFILLKYGLVGGEGGRTTFRLQPPLSQYSVQALRPGSRYEVSVSAVRGTNESDSATTQFTTEIDAPKNLRVGSRTATSLDLEWDNSEAEVQEYKVVYSTLAGEQYHEVLVPRGIGPTTRATLTDLVPGTEYGVGISAVMNSQQSVPATMNARTELDSPRDLMVTASSETSISLIWTKASGPIDHYRITFTPSSGIASEVTVPKDRTSYTLTDLEPGAEYIISVTAERGRQQSLESTVDAFTGFRPISHLHFSHVTSSSVNITWSDPSPPADRLILNYSPRDEEEEMMEVSLDATKRHAVLMGLQPATEYIVNLVAVHGTVTSEPIVGSITTGIDPPKDITISNVTKDSVMVSWSPPVASFDYYRVSYRPTQVGRLDSSVVPNTVTEFTITRLNPATEYEISLNSVRGREESERICTLVHTAMDNPVDLIATNITPTEALLQWKAPVGEVENYVIVLTHFAVAGETILVDGVSEEFRLVDLLPSTHYTATMYATNGPLTSGTISTNFSTLLDPPANLTASEVTRQSALISWQPPRAEIENYVLTYKSTDGSRKELIVDAEDTWIRLEGLLENTDYTVLLQAAQDTTWSSITSTAFTTGGRVFPHPQDCAQHLMNGDTLSGVYPIFLNGELSQKLQVYCDMTTDGGGWIVFQRRQNGQTDFFRKWADYRVGFGNVEDEFWLGLDNIHRITSQGRYELRVDMRDGQEAAFASYDRFSVEDSRNLYKLRIGSYNGTAGDSLSYHQGRPFSTEDRDNDVAVTNCAMSYKGAWWYKNCHRTNLNGKYGESRHSQGINWYHWKGHEFSIPFVEMKMRPYNHRLMAGRKRQSLQF

SEQ ID NO: 3 - DNA, 23 bp, Artificial
  primer
tccatgatcaagccttcagagtg

SEQ ID NO: 4 - DNA, 22 bp, Artificial
  primer
gatgcagccacaggactcaaag

SEQ ID NO: 5 - DNA, 7500 bp, Artificial
  vector pIN-III-ompA2
ggataaccagaagcaataaaaaatcaaatcggatttcactatataatctcactttatctaagatgaatccgatggaagcatcctgttttctctcaatttttttatctaaaacccagcgttcgatgcttctttgagcgaacgatcaaaaataagtgccttcccatcaaaaaaatattctcaacataaaaaactttgtgtaatacttgtaacgctacatggagattaactcaatctagctagagaggctttacactttatgcttccggctcgtataatgtgtggaattgtgagcggataacaatttcacacaggaaacagctatgaccatgattacggattcactggaactctagataacgaggcgcaaaaaatgaaaaagacagctatcgcgattgcagtggcactggctggtttcgctaccgtagcgcaggccgctgaattccaagcttggatccggctgagcaacgacgtgaacgcaatgcgttccgacgttcaggctgctaaagatgacgcagctcgtgctaaccagcgtctggacaacatggctactaaataccgcaagtaatagtacctgtgaagtgaaaaatggcgcacattgtgcgacattttttttgtctgccgtttaccgctactgcgtcacgcgtaacatattcccttgctctggttcaccattctgcgctgactctactgaaggcgcattgctggctgcgggagttgctccactgctcaccgaaaccggataccctgcccgacgatacaacgctttatcgactaacttctgatctacagccttattgtctttaaattgcgtaaagcctgctggcagtgtgtatggcattgtctgaacgttctgctgttcttctgccgatagtggtcgatgtacttcaacataacgcatcccgttaggctccacggaatatttcaccggttcgttgatcactttcaccggcgttcccgtccgcacgctggagaacaaggctttaatatccggtgcattcatgcgaatacaccctgaactgacgcgcaaaccgacgctgtccggcgcactggtaccatgaatgaggtattcgccattaccatgcgcgaggcgcagtgcgtaacgtcctagcgggttatttggtccggcaggaacgactggcggtaatttaatgccacgctccagcgaacgctgacgaatgcctgccgtaggcgtccaggttgggttagggattttctgcccaacacgcgtttccatcaccggcgtttccagcccctgcaatccaatacctattggataaacctgcacaatattttctcccggcggataataataaaggcgcagctctgcaaggttggtcgacggtcgaccggtctcacttcaacataacgcatcccgttaggctccacggaatatttcaccggttcgttgatcactttcaccggcgttcccgtccgcacgctggagaacaaggctttaatatccggtgcattcatgcgaatacaccctgaactgacgcgcaaaccgacgctgtccggcgcactggtaccatgaatgaggtattcgccattaccatgcgcgaggcgcagtgcgtaacgtcctagcgggttatttggtccggcaggaacgactggcggtaatttaatgccacgctccagcgaacgctgacgaatgcctgccgtaggcgtccaggttgggttagggattttctgcccaacacgcgtttccatcaccggcgtttccagcccctgcaatccaatacctattggataaacctgcacaatattttctcccggcggataataataaaggcgcagctctgcaaggttgacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgctaacgataatccctgacgcggtgcatcaggtaataacagttgtgaaggaatagttatcgtcgtaccaggttttggcaccggggcgatagtgttattggcttcaaggatcaacattgccgcagtatcaaaacgtcgggcaatagcctgaaggtttttatccccttcttgcaccgtatacgtttgattttgcccaaccagtcggcttccggttggtggtagcggataatcaaccgcccaggcagcctggatggcgctaaaagcgccgataagcgtgagtgtaagcaaagacgcgcgtttcattgtaaacctcctgtatttgccggagactcacgctgaaacgtcggatggcgcttatgttcacctgaaaccaaaacactcctgtgcaggtcagtgtaaacattgaccatccggcaatgtgagccaaccggatgaaagctgtccttttagtttagctaagtgcagcggctttggcgcgaattgcgcgaatcatcgcttccagaccttgtgaacgagatggggtgagatgttgggtgagcgccattttttcaaaccacggacgcacatcgaaattgacaatatcctgcggcgtcatctgatcgtagagaataaagacgaccgcaataagccctttcacaatcgccgcatcgctgtcgccctgtaattcaataattccctgggcattctggcgcatgacaatccacacctgactctgacagccctgaatgctattttgtggacttctgtcttcgtcgcgtaattctggcagacgctgggggaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcccttt

SEQ ID NO: 6 - protein, 1358 aa, Mus musculus
MGIDGETVVLKNMLIGVNLILLGSMLKPSECRLEVTTERAQRQTVEEEGGASSHNTSSKEQPMVFNHVYNINVPLESLCSSGLEASAEQDMSAEDDTLAEYIGQTSDHESQVTFTHKINLPKKACPCASSSQVLQELLSRIEMLEREVSLLRDQCNTNCCQESAATGQLDYVPHCSGHGNFSFESCGCICNEGWFGKNCSEPYCPLGCSSRGVCVDGQCICDSEYSGDDCSELRCPTDCSSRGLCVDGECVCEEPYTGEDCRELRCPGDCSGKGQCANGTCLCQEGYAGEDCSQRRCLNACSGRGHCQEGLCICEEGYQGPDCSAVAPPEDLRVAGISDRSIELEWDGPMAVTEYVISYQPTALGGLQLQQRVPGDWSGVTIMELEPGLTYNISVYAVISNILSLPITAKVATHLSTPQGLQFKTITETTVEVQWEPFSFSFDGWEISFIPKNNEGGVIAQLPSDVTSFNQTGLKPGEEYIVNVVALKEQARSPPTSASVSTVIDGPTQILVRDVSDTVAFVEWTPPRAKVDFILLKYGLVGGEGGKTTFRLQPPLSQYSVQALRPGSRYEVSISAVRGTNESEASSTQFTTEIDAPKNLRVGSRTATSLDLEWDNSEAEAQEYKVVYSTLAGEQYHEVLVPKGIGPTTKTTLTDLVPGTEYGVGISAVMNSKQSIPATMNARTELDSPRDLMVTASSETSISLIWTKASGPIDHYRITFTPSSGISSEVTVPRDRTSYTLTDLEPGAEYIISITAERGRQQSLESTVDAFTGFRPISHLHFSHVTSSSVNITWSDPSPPADRLILNYSPRDKEEDMLEVLLDATKRHAVLMGLQPATEYIVNLVAVHGTVTSEPIVGSITTGIDPPKNITISNVTKDSLTVSWSSPVAPFDYYRVSYRPTQVGRLDSSVVPNTVTEFAITRLYPATEYEISLNSVRGREESERICTLVHTAMDSPMDLIATNITPTEALLQWKAPMGEVENYVIVLTHFAIAGETILVDGVSEEFQLVDLLPSTHYTVTMYATSGPLMSGTIATNFSTLLDPPDNLTASEVTRQSALISWQPPRAAIENYVLTYKSTDGSRKELIVDAEDTWIRLEGLSENTDYTVLLQAAQEATRSSLTSTVFTTGGRVFSHPQDCAQHLMNGDTLSGVYTIFLNGELSHKLQVYCDMTTDGGGWIVFQRRQNGQTDFFRKWADYRVGFGNLEDEFWLGLDNIHRITAQGRYELRVDMRDGQEAVFAYYDKFAVEDSRSLYKIRIGSYNGTAGDSLSYHQGRPFSTEDRDNDVAVTNCAMSYKGAWWYKNCHRTNLNGKYGESRHSQGINWYHWKGHEFSIPFVEMKMRPYIHRLTAGRKRRALKF

SEQ ID NO: 7 - protein, 1356 aa, Rattus norvegicus
MGIEGETVVLKNMLIGVNLILLGSMLKPSECRLEVTTERVQRQTVEEEGGASSYNTSSKEQPMVFNHVYNINVPLESLCSSGLEASAEQDVSAEDDTLAEYTGQTSDHESQVTFTHKINLPKKACPCASSAQVLQELLSRIEMLEREVSVLRDQCNTNCCQESAATGQLDYVPHCSGHGNFSFESCGCICNEGWFGKNCSEPYCPLGCSSRGVCVDGQCICDSEYSGDDCSELRCPTDCSSRGLCVDGECVCEEPYTGEDCRELRCPGDCSGKGQCANGTCLCQEGYAGEDCSQRRCLNACSGRGHCQEGLCICEEGYQGPDCSAVTPPEDLRVAGISDRSIELEWDGPMAVTEYVISYQPSLGGLQLQQRVPGDWSGVTITELEPGLTYNISVYAVISNILSLPITAKVATHLSTPQGLQFKTITETTVEVQWEPFSFSFDGWEISFTPKNNEGGVIAQLPSDVTSFNQTGLKPGEEYIVNVVALKEQARGPPTSASVSTVIDGPTQILVRDVSDTVAFVEWTPPRAKVDFILLKYGLVGGEGGKTTFRLQPPLSQYSVQALRPGSRYEVSISAVRGTNESDASSTQFTTEIDAPKNLRVGSRTATSLDLEWDNSEAEAQEYKVVYSTLAGEQYHEVLVPKGIGPTTKTTLTDLVPGTEYGVGISAVMNSKQSIPATMNARTELDSPRDLMVTASSETSISLIWTKASGPIDHYRITFTPSSGISSEVTVPRDRTSYTLTDLEPGAEYIISITAERGRQQSLESTVDAFTGFRPISHLHFSHVTSSSVNITWSDPSPPADRLILNYSPRDEEEEMMEVLLDATKRHAVLMGLQPATEYIVNLVAVHGTVTSEPIVGSITTGIDPPKNITISNVTKDSLTVSWSPPVAPFDYYEYPIDHPSGRLDSSVVPNTVTEFTITRLYPASQYEISLNSVRGREESERICTLVHTAMDSPMDLIATNITPTEALLQWKAPMGEVENYVIVLTHFAMAGETILVDGVSEEFQLVDLLPRTHYTVTMYATSGPLVSGTIATNFSTLLDPPANLTASEVTRQSALISWQPPRAAIENYVLTYKSTDGSRKELIVDAEDTWIRLEGLSENTDYTVLLQAAQEATRSSLTSTIFTTGGRVFSHPQDCAQHLMNGDTLSGVYTIFLNGELSHKLQVYCDMTTDGGGWIVFQRRQNGQTDFFRKWADYRVGFGNLEDEFWLGLDNYHRITAQGRYELRVDMRDGQEAVFAYYDKFAVEDSRSLYKLRIGGYNGTAGDSLSYHQGRPFSTEDRDNDVAVTNCAMSYKGAWWYKNCHRTNLNGKYGESRHSQGINWYHWKGHEFSIPFVEMKMRPYIHRLTAGRKRRALKF

SEQ ID NO: 8 - protein, 1334 aa, Homo sapiens
MIKPSECQLEVTTERVQRQSVEEEGGIANYNTSSKEQPVVFNHVYNINVPLDNLCSSGLEASAEQEVSAEDETLAEYMGQTSDHESQVTFTHRINFPKKACPCASSAQVLQELLSRIEMLEREVSVLRDQCNANCCQESAATGQLDYIPHCSGHGNFSFESCGCICNEGWFGKNCSEPYCPLGCSSRGVCVDGQCICDSEYSGDDCSELRCPTDCSSRGLCVDGECVCEEPYTGEDCRELRCPGDCSGKGRCANGTCLCEEGYVGEDCGQRQCLNACSGRGQCEEGLCVCEEGYQGPDCSAVAPPEDLRVAGISDRSIELEWDGPMAVTEYVISYQPTALGGLQLQQRVPGDWSGVTITELEPGLTYNISVYAVISNILSLPITAKVATHLSTPQGLQFKTITETTVEVQWEPFSFSFDGWEISFIPKNNEGGVIAQVPSDVTSFNQTGLKPGEEYIVNVVALKEQARSPPTSASVSTVIDGPTQILVRDVSDTVAFVEWIPPRAKVDFILLKYGLVGGEGGRTTFRLQPPLSQYSVQALRPGSRYEVSVSAVRGTNESDSATTQFTTEIDAPKNLRVGSRTATSLDLEWDNSEAEVQEYKVVYSTLAGEQYHEVLVPRGIGPTTRATLTDLVPGTEYGVGISAVMNSQQSVPATMNARTELDSPRDLMVTASSETSISLIWTKASGPIDHYRITFTPSSGIASEVTVPKDRTSYTLTDLEPGAEYIISVTAERGRQQSLESTVDAFTGFRPISHLHFSHVTSSSVNITWSDPSPPADRLILNYSPRDEEEEMMEVSLDATKRHAVLMGLQPATEYIVNLVAVHGTVTSEPIVGSITTGIDPPKDITISNVTKDSVMVSWSPPVASFDYYRVSYRPTQVGRLDSSVVPNTVTEFTITRLNPATEYEISLNSVRGREESERICTLVHTAMDNPVDLIATNITPTEALLQWKAPVGEVENYVIVLTHFAVAGETILVDGVSEEFRLVDLLPSTHYTATMYATNGPLTSGTISTNFSTLLDPPANLTASEVTRQSALISWQPPRAEIENYVLTYKSTDGSRKELIVDAEDTWIRLEGLLENTDYTVLLQAAQDTTWSSITSTAFTTGGRVFPHPQDCAQHLMNGDTLSGVYPIFLNGELSQKLQVYCDMTTDGGGWIVFQRRQNGQTDFFRKWADYRVGFGNVEDEFWLGLDNIHRITSQGRYELRVDMRDGQEAAFASYDRFSVEDSRNLYKLRIGSYNGTAGDSLSYHQGRPFSTEDRDNDVAVTNCAMSYKGAWWYKNCHRTNLNGKYGESRHSQGINWYHWKGHEFSIPFVEMKMRPYNHRLMAGRKRQSLQF

SEQ ID NO: 9 - protein, 1353 aa, Gallus gallus
MGTDSENPVLRNVLISFNLLLLGAVLKPFECRLEVTTEPAERPAVDEEGGLANCSPPVKEQPMVFHHIYNINVPVDSCCSSMLRSSAEEVSSEDDRLAEYTEQTSDSESQVTFTHRINLPKQACKCSTSLPSLQELLSRIEMLEREVSMLRDQCNSNCCQENAATGRLDYTLPCSGHGNFSLESCRCICSEGWAGSNCSEPRCPRGCSSRGVCLEGQCVCDNDYGGEDCSQLRCPAGCGSRGLCVDGECICEEGFGGEDCSQPRCPRDCSGRGHCDNGTCVCAEGYAGEDCGWLRCPNACSGRGVCQDGLCICEDGYGGQDCSAVAPPENLRVTGISDGSIELAWDSLGAATEYVVSYQPAGPGGSQLQQRVPGDWSTITITELEPGVAYNVSIYAVISDVLSSPVTTKVTTNLATPQGLKFKTITETTVEVQWEPFSFPFDGWEISFIPKNNEGGVIAQLPSTVTTFNQTGLKPGEEYTVTVVALKDQARSPPASDSISTLIDGPTQILVRDVSDTVAFVEWTPPRARVDAILLKYGLADGEGGRTTFRLQPPLSQYSLQALRPGARYHLAVSALRGANESQPALAQFTTEIDAPKNLRVGSRTPASLELTWDNSEAEAHSYRVVYSTLAGEHYHEVLVPRDTGPTTRATLADLVPGTEYGIGISAVMDSQQSVPATMNARTELDSPRDLLVTASTETSISLSWTKAMGPIDHYRVTFTPASGMASEVTVSRNESQLTLSELEPGTEYTISIIAERGRQQSLEATVDAFTGVRPITQLHFSQLTSSSVNITWSDPSPPADRLVLTYSPRDEEAPQQLALDGTRRHASLTGLRPSTEYLVSLVAVHGAVSSEPVTGSITTGMDAPKDLRVGNITQDSMVIYWSPPVAPFDHYRISYRAAEGRTDSTAIGNDATEYIMRLLQPATKYEIGVKSVRGREESEVASITTYTAMDAPLGVTATNITPTEALLQWNPPLMDVESYVLVLTRHTGETILVDGINQEYQLTNLQPSTTYTVAMYATNGPLTSQTISTNFTTLLDPPTNLTASEVTRRSALLSWVPPVGDIENYILTYRSTDGSRKELIVDAEDTWIRLEGLSETTQYTVRLQAAQNAMRSGFISTTFTTGGRVFANPQDCAQHLMNGDTLSGVYTISINGDLSQRVQVFCDMSTDGGGWIVFQRRQNGLTDFFRKWADYRVGFGNLEDEFWLGLDNIHKITSQGRYELRIDMRDGQEAAYAYYDKFSVGDSRSLYKLRIGDYNGTSGDSLTYHQGRPFSTKDRDNDVAVTNCAMSYKGAWWYKNCHRTNLNGKYGESRHSQGINWYHWKGHEFSIPFVEMKMRPYNHRNISGRKRRSLQL

SEQ ID NO: 10 - protein, 1350 aa, Danio rerio
MWGCAMAIQGSVVSLALLFFGIQAVPSPTSKLVRTTRVRRQVPEGGDPADNQMQPMVFNHVYNINVPVESLCSVDLDAVTGPETNKTASGSDKMPTEYTEETVDSDSQVTFTHRINIPKQACACPSATTIEQLASRIEMLEREVSLLRTQCSSSCCGESSVMGRLDFVPQCGHGTFSMEVCGCVCEEGWIGKNCTEPRCPDDCSGQGICIEGDCVCDRNFGGENCSEPRCPSDCSDRGLCIDGECVCEEAFAGEDCSLGRCLNDCSDQGACVNGSCQCRSGFLGEDCSLIFCANNCSQRGVCKEGFCVCQEGYTGDDCTSVLPPMNLRVRGVSENTIDLQWEGPAILTDYLITYAPTTPGGVQLEMRVPGNVTNITIKGLNPGLEYNVNVYAVIDNIISSPMNTLVSTYLSNPDGLLFKSIMETSVEVQWQPLDYLFDGWEISFIPKDNDGGMTAQLPSTITSFHQTGLKPGEEYTVNLVALKDQGRSLPVTATVTTLIDGPIQLIVRDISDTVAFVEWSPPKAKVDQIILRYGLVGVEGPKTTFRLQPTLSQYSLQVLRPGSTYEVSVSGVRNGTESGVISTEFTTEIDAPKNLRVLSKTSISLELEWDNSEADVEGYSVVYSTLAGDQYDKVIVPRNDGPSTKTTLNELMPGTEYGIGISAVLGANQSTPATMNARTGLDVPLDLTVTASTDNTITLLWGTVQGPIDHYRVTYTSSSGVTTELTVPKDVTTTTLTSLEPGTKYTITVAAQRGRQQSTAATIDAFTGFRPITQLFFSEVSSDSLTVAWSSPAPPADAFILNYSAQDSSEDSEIALDGSKTRITLTGLMPSRRYTVTLVTMHGNVTSKPVVGSVNTGMDPPRDITVLYVTEESVTITWIQPLALFDYYRMSYQSSKGRMDSVVIDNDINNYTLTSLHPATEYEIKLNAVRGSQESKVITTTVFTAMDMPAELTALNVTPQGALLRWNPPASSVDNYVLTVTRNQVTADTFLVEGNKQEYQLSQLIPSTTYSVALYATKGPLTSGTVISNFVTPMDAPQNLTASEINHRSALISWQPPIADIDNYMLTYKAADGSRKELILDAEDTWIRLEGLAETTEYTVRLQAARGLETSAIVSTSFTTGNRLFPTPQNCAQHLLNGETLGGIYTIYVNRDLSQGVQVYCDMTTDGGGWIVFQRRQNGLTDFSRKWTDYKIGFGNLEDEFWLGLDNIHKIAAQGRYELRIDMKDGQESVYANYDRFSIGDSKSLYKLRIGEYNGTAGDSLSYHQSRPFSTKDKDNDIAVTNCALSYKGAWWYKNCHRANLNGKYGESRHSQGINWYHWKGHEFSIPFVEMKMRPFNYRSISGKRRRSTH